(19) United States
(12) Patent Application Publication
(10) Pub. No.: US 2026/0234215 A1
(43) Pub. Date: Aug. 13, 2026

(54) Anti-Inflammatory Bio-Active Collagen Hybridizing Peptides and Methods of Using Thereof

(71) Applicant: 3HELIX, INC., Salt Lake City, UT (US)

(72) Inventors: Michael KIRKNESS, Salt Lake City, UT (US); Lucas BENNINK, Salt Lake City, UT (US)

(73) Assignee: 3HELIX, INC., Salt Lake City, UT (US)

(21) Appl. No.: 19/156,875

(22) PCT Filed: Mar. 1, 2024
(86) PCT No.: PCT/US2024/018078

§ 371 Date: Aug. 14, 2025

Related U.S. Application Data
(60) Provisional application No. 63/449,575, filed on Mar. 2, 2023.

(51) Int. Cl. C07K 14/705; A61K 38/00; A61K 47/64; A61P 17/00; A61P 17/06; A61P 27/02; A61P 37/06

(52) U.S. Cl. CPC C07K 14/70503; A61K 47/6435; A61P 17/00; A61P 17/06; A61P 27/02; A61P 37/06; C07K 14/70535; A61K 38/00

(57) ABSTRACT

Novel anti-inflammatory bioACTIVE collagen hybridizing peptides (CHPs), compositions comprising the CHPs, and methods of using the CHPs and the CHP-comprising compositions are disclosed. The novel anti-inflammatory bioACTIVE CHPs can be used to localize collagen anti-inflammatory sequences to directly downregulate the inflammatory response.

### BACKGROUND

Collagen homeostasis is a complex process which involves both the synthesis and degradation of collagen. Collagen homeostasis is tightly controlled mainly via a specialized group of cells called fibroblasts. Fibroblasts produce both collagen and matrix metalloproteinases (MMPs) in response to chemical signals, in response to cell to extracellular matrix (ECM) interactions, and in response to environmental conditionals like UV light and force. Collagen is the major structural protein found in almost all human tissues. Collagen has binding sequences present that are required to be in triple helical form to be recognized by cells including fibroblasts and other types including immune cells. Cells bind to collagen through cell surface receptors including integrins, discoidin domain receptors, MMP, osteoclast-associated receptors (OSCAR), Leukocyte associated immunoglobulin like receptor 1 (LAIR-1) and many others. Importantly immune cell activity can be mediated through their interactions with triple helical collagen through cell surface LAIR-1.

It has been shown that degraded collagen is present in damaged tissues and is often associated with many human diseases and injuries. Damaged collagen lacks the triple helical structure that allows for recognition by fibroblasts, other cell types, or biological molecules (e.g., MMPs). Sites of damaged collagen often additionally exhibit inflammatory and auto-immune activity (e.g., an overabundance of inflammatory cytokines), which can further damage collagen and the ECM. Collagen damage leads to a poor microenvironment for cell adhesion and mechanics which can cause fibroblasts to decrease their production of collagen and leave the affected area susceptible to further degradation. This may occur under different conditions, including in diseases like osteoarthritis; as a result of natural events like aging (senescent fibroblast aging); under decreased mechanical stimulation; under exposure to environmental factors like UV damage; or under exposure to chemical signals like reactive oxygen species. Conditions such as dry-eye disease (DED), atopic dermatitis (eczema), psoriasis, diabetic lesions, osteoarthritis, rheumatoid arthritis, and other inflammatory disorders may all be accompanied by both damaged collagen which can lead to a failure in barrier function and an excessive inflammatory response near the site of collagen damage.

One may address both a poor cell microenvironment and the increased inflammatory response by providing localized triple helical binding sites for cells to bind to at the sites of increased collagen damage. Offering new binding sites to the microenvironment will increase collagen production thereby increasing mechanical stimulation for the cells in order to create a positive feedback loop. More native collagen equates to increased binding sites and mechanical stimulation, which can encourage tissue regeneration and repair from resident cells. Moreover, increasing collagen anti-inflammatory sequences at the site of collagen damage will help reduce or prevent the activation of immune cells, including but not limited to T-Cells, natural killer cells, macrophages, B Cells, neutrophils, and monocytes. Therefore, it would be beneficial to repair damaged collagen-based, cellular environments with methods and compositions which can stimulate fibroblasts to increase collagen production, increase barrier function, and downregulate the immune response to restore structural integrity of the ECM and ultimately restore tissue pathologies to a healthy state.

### SUMMARY OF THE DISCLOSURE

The present disclosure relates to novel anti-inflammatory bioACTIVE collagen hybridizing peptides (CHPs), compositions comprising the CHPs, and methods of using the CHPs and the CHP-comprising compositions. The novel anti-inflammatory bioACTIVE CHPs can be used to localize collagen anti-inflammatory sequences to directly downregulate the immune and inflammation response. The disclosure of PCT/US2022/077730 is hereby incorporated by reference for all purposes herein.

### BRIEF DESCRIPTION OF THE DRAWINGS

FIG. 1A illustrates a bioactive CHP, comprising of triple helical forming sections covalently linked to the bioactive sequence(s).

FIG. 1B represents the unique binding mechanism of the bioactive CHP. This allows the CHP to be inactive in its single chain conformation but becomes activated once it forms a triple helix with the damaged collagen. Thus, the bioactive CHP is capable of targeting and localizing to remodeling or damaged collagen. Once bound to the damaged collagen, it forms the appropriate triple helix and presents the bioactive sequence in the correct conformation for recognition, thereby facilitating therapeutic action through interaction with cells recognizing the bioactive sequence to promote ECM healing and/or regeneration.

FIG. 2 depicts the numerous immune cells that have a LAIR-1 receptor and can interact with a naturally occurring collagen molecule that is in a triple-helical conformation. The proposed bioACTIVE CHP can incorporate a LAIR-1 bioactive sequence and present the LAIR-1 binding site in the correct conformation upon CHP binding with damaged collagen for recognition and binding by immune cells to reduce inflammatory response.

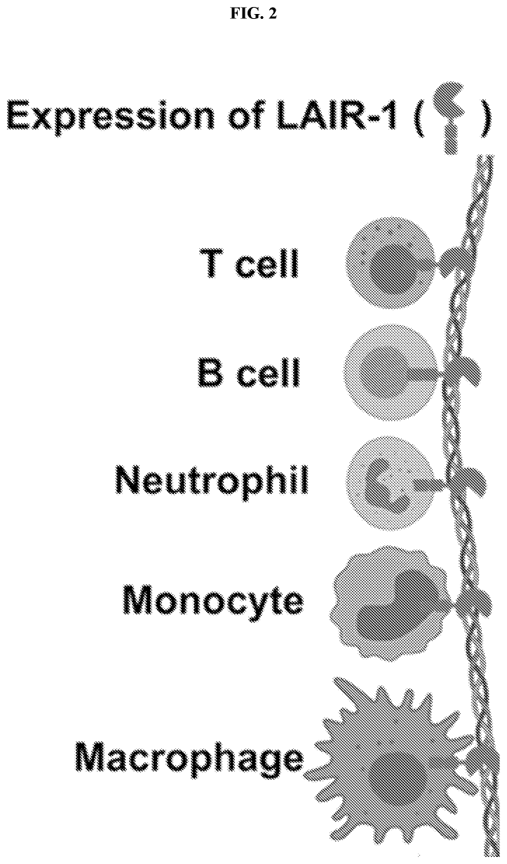

FIG. 3 depicts a bioactive CHP in monomeric form (with a monomeric binding site), a dimeric bioactive CHP (two linked bioactive and folding regions), and a bioactive CHP bound to two other CHPs in a trimeric format (presenting a trimeric binding site) with a monomeric “tail” portion of the bioactive CHP in accordance with embodiments disclosed herein.

FIG. 4 depicts the proposed mechanism of action for LAIR-1 bioactive CHPs in accordance with embodiments disclosed herein.

FIG. 5 depicts exemplary bioactive CHPs amino acid sequences in accordance with embodiments disclosed herein.

FIG. 6 depicts the experimental design of a LAIR-1 plate binding assay to detect LAIR-1 binding to LAIR-1 monomeric bioactive CHPs in accordance with embodiments disclosed herein.

FIG. 7 depicts the results of a plate binding assay at room temperature for a LAIR-1 monomeric bioactive CHP with a high melting temperature in accordance with embodiments disclosed herein.

FIG. 8 depicts the results of a plate binding assay at 37° C. for a LAIR-1 monomeric bioactive CHP with a high melting temperature in accordance with embodiments disclosed herein.

FIG. 9 depicts the results of a plate binding assay at 4° C. for a LAIR-2 monomeric bioactive CHP with a low melting temperature in accordance with embodiments disclosed herein.

FIG. 10 depicts the experimental design of a LAIR-1 plate binding assay to detect LAIR-1 binding to LAIR-1 dimeric bioactive CHPs in accordance with embodiments disclosed herein.

FIG. 11 depicts the results of a binding assay at 4° C. for a LAIR-1 dimeric bioactive CHP with a low melting temperature in accordance with embodiments disclosed herein.

FIG. 12 depicts an experimental design for demonstrating T-cell activation/inhibition in vitro using LAIR-1 CHPs in accordance with embodiments disclosed herein.

FIG. 13 depicts experimental conditions used in the assay for demonstrating T-cell activation/inhibition in vitro using LAIR-1 CHPs in accordance with embodiments disclosed herein.

FIG. 14 depicts the results of an in vitro T-cell activation assay at 37° C. via detection of IL-2 levels 72 hours post-stimulation in accordance with embodiments disclosed herein.

FIG. 15 depicts the results of an in vitro T-cell activation assay at 37° C. via detection of IL-2 levels 72 hours post-stimulation in accordance with embodiments disclosed herein.

### DETAILED DESCRIPTION

Hereinafter, exemplary embodiments of the present disclosure will be described in detail. However, the present disclosure is not limited to the embodiments disclosed below, but may be implemented in various forms. The following embodiments are described in order to enable those of ordinary skill in the art to embody and practice embodiments of the present disclosure.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide conjugate is disclosed and discussed and a number of modifications that can be made to a number of molecules including the peptide conjugate are discussed, each and every combination and permutation of the peptide conjugate and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### Definitions

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

It must be noted that as used herein and in the appended claims, the singular forms “a”, “an”, and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth. Similarly, the word “or” is intended to include “and” unless the context clearly indicates otherwise.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary unless a particular order is clearly indicated by the context.

As used herein, the term “about” refers to a numeric value, including, for example, whole numbers, fractions, and percentages, whether or not explicitly indicated. The term “about” generally refers to a range of numerical values (e.g., +/−5, 6, 7, 8, 9 or 10% of the recited value) that one of ordinary skill in the art would consider equivalent to the recited value (e.g., having the same function or result). In some instances, the term “about” may include numerical values that are rounded to the nearest significant figure. In general, the term “about” includes at least the values for a given quantity that fall within the corresponding tolerances for manufacture, formulation and/or measurement.

As used herein “collagen” can be from any tissue type (e.g., bone, dermis, tendon, ligaments, cornea etc.). Collagen can refer to a molecule in which three alpha chains of polyproline II-like structure fold together into a triple helix. Additionally, this can apply to any protein that contains a triple-helical region including collagen types I-XXVIII and bacterial collagen. The term “collagen” as used herein can refer to all forms of collagen, including artificial collagen and collagen which has been processed or otherwise modified. In some embodiments, the collagen is selected from type I collagen, type II collagen, type III collagen, type IV collagen, type V collagen, type VI collagen, type VII collagen, type VIII collagen, type IX collagen, type X collagen, type XI collagen, type XII collagen, type XIII collagen, type XIV collagen, type XV collagen, type XVI collagen, type XVII collagen, type XVIII collagen, type XIX collagen, type XX collagen, type XXI collagen, type XXII collagen, type XXIII collagen, type XXIV collagen, type XXV collagen, type XXVI collagen, type XXVII collagen, type XXVIII collagen, and a combination thereof.

As used herein, the term “proline or modified proline” means the amino acid proline and various isomers, analogs and variants thereof, including both natural and non-natural isomers. In one example, the modified proline includes an electron withdrawing group. Examples of modified proline include, without limitation, hydroxyproline, methylated proline, 4-fluoroproline, and 4-chloroproline.

The term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” a disease or injury involving collagen damage can refer to reducing or eliminating the amount of damaged/denatured collagen. Treatment can also be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

### Introduction

With reference to the appended drawings, exemplary embodiments of the present disclosure will be described in detail below. To aid in understanding the present disclosure, like numbers refer to like elements throughout the description of the figures, and the description of the same elements will be not reiterated.

Collagen is a major structural protein found in almost all human tissues. Degraded collagen has been shown to be present in damaged tissues and is often associated with many human diseases and injuries—including inflammatory conditions and auto-immune disorders.

Intact collagen which is found within the ECM is present as a triple-helical molecule. Cells are capable of binding to intact collagen at specific sequences/sites, and this binding helps to maintain the health and structures of the ECM and resident cells. When collagen is damaged, it loses its triple helicity which changes the structure of the cell binding sites and ultimately limits the cell's ability to bind to the collagen. When immune cells cannot bind to collagen, which acts as a natural check point inhibitor, it can increase the presence of matrix metalloproteinases (MMPs) and inflammatory cytokines at the site of the damage. This results in a negative feedback loop, wherein the MMPs and inflammatory cytokines prevent tissue repair, and subsequent collagen damage can further increase the presence of MMPs and inflammatory cytokines.

Collagen hybridizing peptides (CHP) are short repeating Gly-X-Y tripeptides capable of specifically binding to degraded collagen with little to no affinity for intact collagen molecules. CHPs can distinguish damaged collagen by recognizing the structural motif of the poly-proline II helix of individual alpha chains which are not available on intact collagen molecules; upon identifying the damaged collagen the CHPs can then re-form and repair the collagen triple helix through hybridization with the damaged collagen, thereby forming highly stable and long-lasting bonds. The present disclosure relates to bioACTIVE versions of CHPs that can be used to induce cells to produce more collagen, but also to localize collagen based anti-inflammatory cell binding sequences to directly downregulate the inflammatory response in damaged tissues.

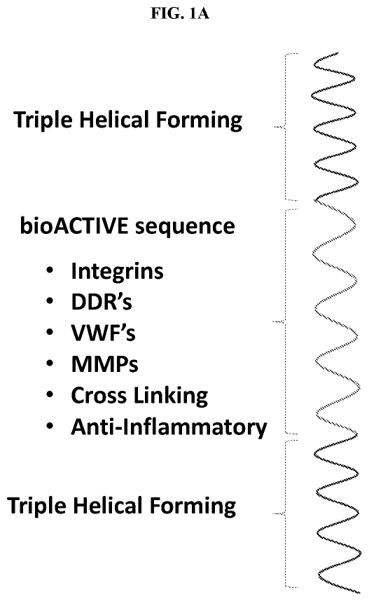

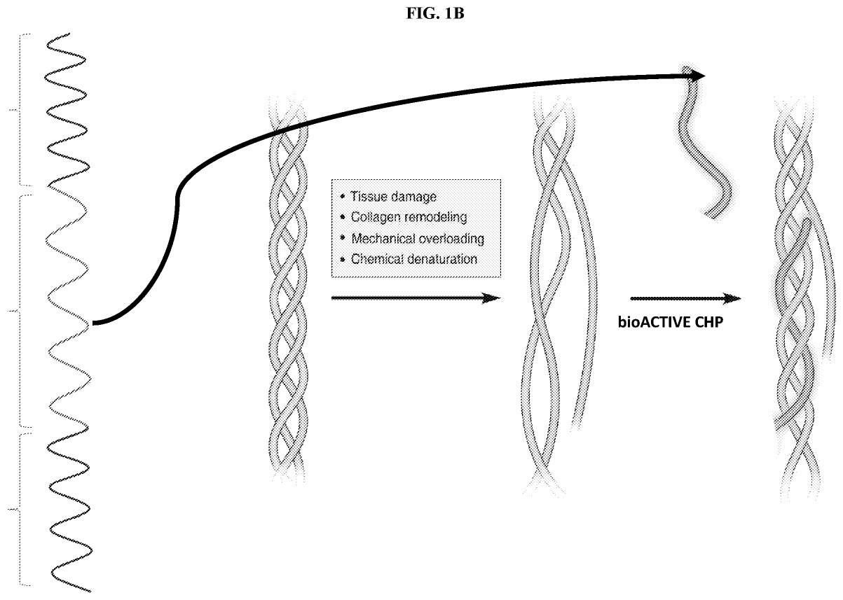

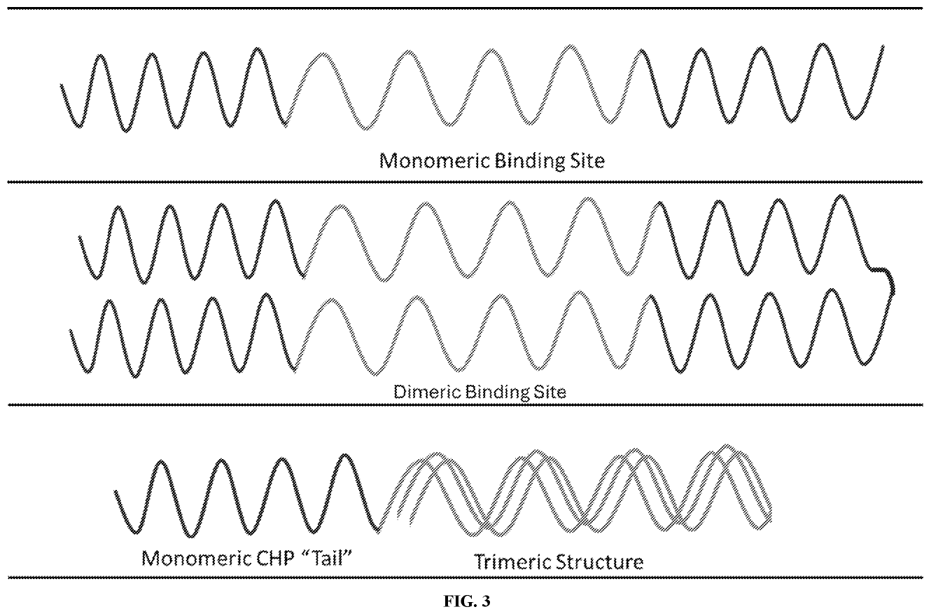

Bioactive CHPs generally include 3 primary segments: a triple helix-forming segment, followed by a bioactive sequence (e.g., a cell binding sequence), followed by a second triple helix-forming segment (an exemplary diagram of a bioactive CHP is set forth at FIG. 1A). These CHPs can be in monomeric, dimeric, or trimeric forms depending on the need of the biological receptor to which the bioactive sequence is intended to bind (see FIG. 3). Dimeric and trimeric forms of CHPs may include a linker between CHP strands. Such linkers may include 6-aminohexanoic acid and SEQ ID NO: 105 (wherein z is any integer between 1 and 5). These CHPs target areas of damaged collagen by localizing to and hybridizing with damaged or remodeling areas of collagen to reform the appropriate triple helix structure. Upon localization, the formation of the triple helix permits the CHP to perform its therapeutic action via presentation of the bioactive sequence in the correct conformation for binding. The proposed localization mechanism of action for exemplary bioactive CHPs is depicted in FIG. 1B.

The inflammatory/immune response can be downregulated, inhibited, or reduced in a number of ways by administering the CHPs and/or compositions of the present disclosure. Inflammation can also be reduced, prevented, or treated using the CHPs and/or compositions of the present disclosure. Immune related cytokines (e.g., TLR ligands, interleukins, IFN-α, TLR-9, TNF-α, IL-1β, IL-6, CCL3, IL-16, IL-18, and/or TNF-α) can be downregulated using embodiments of the disclosed CHPs and/or compositions. Embodiments of the disclosed CHPs and/or compositions can also prevent immune cell differentiation (e.g., T-Cells, B-Cells, Natural Killer Cells, Macrophages, Monocytes, Neutrophils). Other embodiments of the disclosed CHPs and/or compositions can downregulate immune cell activity (e.g., T-Cells, B-Cells, Natural Killer Cells, Macrophages, Monocytes, Neutrophils).

The anti-inflammatory bioactive CHP sequences of the present disclosure leverage specific amino acid sequences that are taken from anti-inflammatory peptide sequences (for example, LAIR-1 receptors) that cells recognize and respond to, ultimately reducing the inflammatory response. These sequences act as natural checkpoint inhibitors. By adding these sequences into the CHP, the biological functions that natural, undamaged collagen offers (such as cell binding and ECM-cell signaling) can be recaptured in a novel manner that involves the CHPs folding into triple-helical conformation with damaged or denatured collagen strands. The bioactive sequences that are inserted into the CHPs can then be presented in the correct conformation to attract and bind to cells in the ECM, to downregulate inflammatory response, and to help treat chronic wounds or disease states that involve damaged collagen. Such CHPs and compositions including them can be used for a number of different diseases or conditions characterized by damaged collagen and inflammatory response (e.g., DED, atopic dermatitis, osteoarthritis, rheumatoid arthritis, and diabetic lesions).

CHPs and their compositions provide a novel way of localizing anti-inflammatory sequences in the correct, triple-helical conformation while maintaining high solubility in water. Due to their high solubility, low immunogenicity, and small size, anti-inflammatory bioactive CHPs can be implemented in several formulations without difficulty for use in addressing a number of different conditions or disorders. The anti-inflammatory bioactive CHPs and their compositions disclosed herein offer improvements over current anti-inflammatory treatments for conditions, as the disclosed CHPs can localize to the area that has the most inflammation and collagen turnover. Once bound to the damaged collagen strand, the anti-inflammatory CHP amino acid sequence will be in the correct triple-helical conformation to be recognized by pertinent inflammatory or ECM resident cells. Depending on the specific sequence used, the anti-inflammatory bioactive CHPs can either increase the collagen repair to help restore barrier function of the tissue, inhibit the inflammatory response, or both.

By treating both the inflammatory and poor cell environment, the speed of the recovery should be improved greatly. Additionally, due to the long binding time of CHPs (e.g., greater than 5 days) patients will not have to apply the therapeutic including the anti-inflammatory bioactive CHPs as frequently; thus, patient compliance will increase. This is especially true in disease cases where traditional treatments are typically needed multiple times a day, including but not limited to DED and atopic dermatitis. The CHPs and compositions including them can be formulated for use in topical cream formulations, saline solutions (e.g., eye drops), and for systemic injection.

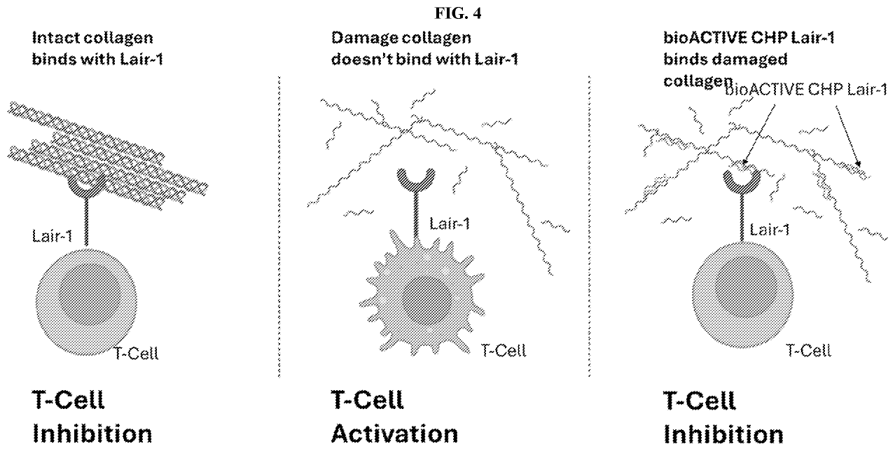

LAIR-1 is a known down-regulator of the T-Cell mediated inflammatory pathway. LAIR-1 is an immune inhibitory receptor that is broadly expressed across immune cells, including but not limited to, T-cells, B-cells, monocytes, neutrophils, natural killer cells and macrophages. When cells expressing LAIR-1 bind an intact collagen LAIR-1 binding site there is a down regulation of immune cell activity and inflammatory cytokines including NF-κB and IFN-γ. However, when there is increased damaged collagen as is found in diseases such as DED, AD, and OA, the inflammatory response is increased due to the lack of the collagen binding sites for LAIR-1 found on immune cells. That is, LAIR-1 does not bind with damaged (unfolded) collagen, which allows T-cells to activate. Additionally, there is a potential negative feedback loop as active immune cells are known to produce MMPs which further degrade the surrounding collagen which cleaves any remaining LAIR sites. A diagram of T-Cell inhibition by intact collagen, T-Cell activation in the environment of damaged collagen, and proposed mechanism for T-cell inhibition using exemplary LAIR-1 bioactive CHPs as disclosed herein is shown in FIG. 4.

Without being bound by theory, it is believed that LAIR bioactive CHPs will decrease immune activation by creating more potential binding sites for immune cells (such as T-cells) that have LAIR-1 on their cell surfaces. CHPs allow the LAIR-1 receptor to bind to triple helical collagen sites even in areas of damaged or remodeling collagen. Damaged collagen is upregulated locally in diseases areas in inflammatory, autoimmune, cancer, and fibrotic conditions.

LAIR exists in two states, cell membrane-bound (LAIR-1) and soluble (LAIR-2). The structural difference between the two variants is primarily that LAIR-2 lacks the transmembrane and cytoplasmic regions found on the membrane-bound LAIR-1, allowing it to be excreted extracellularly. LAIR-1 and LAIR-2 act as competitors for the same sites on triple helical collagen, with only approximately 10 sites per collagen molecule available in areas with high GPO content. LAIR-2 is thought to have slightly higher affinity for the sites on collagen than LAIR-1, thereby allowing it to preferentially bind.

Increased LAIR-2 concentration is correlated with an increased survival rate in some cancers. Additionally, the synovial fluid of rheumatoid arthritis (RA) patients has been shown to have higher levels of LAIR-2 as compared to patients with osteoarthritis, and elevated levels of LAIR-2 have been documented in some tissue-specific autoimmune conditions like Graves' disease. This suggests LAIR-2 is increased in inflammatory and autoimmune conditions.

By providing an increased number of LAIR binding sites on damaged collagen, it is believed that bioactive CHPs can help provide additional sites for immune cells expressing LAIR-1 to bind despite LAIR-2's preferential binding, thereby helping to overcome the “blocking” characteristics of LAIR-2. Moreover, since LAIR-2 is a homolog of LAIR-1 and binds to the same site on intact collagen, the bioactive CHPs with LAIR binding sites are thought to be able to bind and sequester excess LAIR-2 around damaged collagen.

In inflammatory conditions (e.g., rheumatoid arthritis, Graves' disease) there is increased damage to collagen, which reduces the number of potential LAIR binding sites as the collagen must be in the appropriate triple helical conformation to present the LAIR binding sites. These inflammatory conditions are also characterized by increased LAIR-2 production, which results in out-of-control inflammatory response. Without being bound by theory, it is believed an inflammatory condition in which LAIR-2 production is upregulated may be treated with administration of bioactive CHPs with LAIR binding sites; such administration would increase the total number of LAIR binding sites available in areas of damaged or remodeling collagen. By increasing the total number of LAIR binding sites, such CHPs will act as a “sink” for the soluble LAIR-2 molecules, reducing the competition for the LAIR sites. This will provide the opportunity for cell-bound LAIR-1 to bind to collagen, reducing the inflammatory response.

### Inflammatory Conditions

A substantial number of diseases, disorders, and conditions involve inflammation in conjunction with collagen damage; thus, treatment of these diseases would benefit from an interruption of the negative feedback loop where increased collagen damage leads to increased inflammatory response which in turn causes more collagen damage, via the enrichment of the damaged collagen with bioactive sites eventual repair of the collagen/ECM in conjunction with the reduction of inflammation. The disclosed CHPs and their compositions can be used in the clinical setting for conditions such as DED, atopic dermatitis, osteoarthritis, rheumatoid arthritis, and diabetic lesions, among others.

### Dry-Eye Disease

Dry-eye disease (DED) is a commonly occurring ocular disorder, affecting up to 50% of the population of all ages. DED is characterized by corneal thinning, nerve damage, cell death, poor tear quality, and an inflammatory response that can be quite robust. This can lead to blurred vision or even vision loss and can be a painful chronic condition for patients to manage.

Currently, there are over-the-counter (OTC) lubricants available to help with the pain for those with mild DED. The problem with the OTC solutions is that they do nothing to address the underlying causes of DED and only provide temporary relief by increasing the moisture content as lubrication.

Most lubricating eye drops are basic saline solutions, but others like iVIZIA can be more advanced. iVIZIA uses hydrating polymers, sodium hyaluronate, and trehalose (a naturally occurring disaccharide that protects from drying out). These types of treatments do not reverse or address the root cause of DED and would be helpful only to patients with mild to moderate DED.

Another treatment option for DED utilizes corticosteroids. EYSUVIS by Kala Pharmaceuticals is a corticosteroid treatment for DED that has been designed with a surface coating to help deliver the steroids to the surface of the eye without getting caught in the protective mucins. This helps to locally decrease the swelling associated with DED, which in turn helps to reduce redness, irritation, and blurry vision. However, this is only good for short term relief and can only be used for a short duration (i.e., only two weeks). Moreover, this treatment fails to address the underlying mechanism of DED inflammation—it only treats the symptoms.

For more severe cases of DED, some treatments aim to stop inflammatory cells from adhering and producing more cytokines and MMPs that will exacerbate the condition. XIIDRA (lifitegrast) by Novartis, works by blocking the LFA-1 receptor on T-cells so they cannot bind to ICAM-1 receptor on other leukocytes and endothelial cells. By this mechanism, they reduce the number of activated T-cells as well as prevent their migration to the eye surface thus offer a way to reduce the inflammatory response. This treatment requires patients to take twice-daily doses for many weeks before seeing a benefit, and patients require continual doses to maintain the effect.

DED patients prescribed an anti-inflammatory regimen typically require multiple uses over longer periods of time to realize any benefit. In order to effectively counteract the inflammatory conditions typically present within ocular tissues as a result of DED, there is a need to address both the breakdown of collagen and to localize delivery of anti-inflammatory signals at the site of lesions. Additionally, anti-inflammatory treatments ignore the poor cell microenvironment and barrier function breakdown.

Anti-inflammatory bioactive CHPs according to the present disclosure offer a novel method of localizing anti-inflammatory sequences in the correct, triple-helical conformation of native collagen while maintaining high solubility in water, which will aid the formulation of treatment compositions such eye drops. Moreover, due to their small size and the reduced barrier function related to DED, CHPs can cross the epithelial barrier of the cornea which is crucial for developing treatment compositions (e.g., eye-drops) and reaching inflamed lesions on the surface of the eye. Such CHPs directly target the unstructured collagen strands found in areas where corneal thinning or corneal ulcers have developed due to the presence of inflammatory cytokines and matrix metalloproteinases (MMPs). This permits the anti-inflammatory bioactive CHPs to target the most irritated regions, hybridize with the denatured collagen, and remain bound for extended periods of time (up to several days) which will allow the cells in the region to interact with them down regulate the inflammatory response and increase the collagen production. The long residence time at the site of damage means that patients will not have to apply drops frequently (e.g., twice daily), but instead only apply once per week, which will likely increase patient compliance. The anti-inflammatory bioactive CHPs may also be combined with other types of bioACTIVE CHPs such integrins and DDRs designed to increase cell migration into the affected areas and increase the collagen production, thereby reducing the inflammatory response, restoring barrier function, and repairing local damage.

### Atopic Dermatitis

Atopic dermatitis (i.e., “eczema”) is an inflammatory, T-cell mediated, chronic skin condition that causes dry, itchy, and inflamed skin. It is very common in youth, especially in babies. The Atopic Dermatitis first line of treatments are moisturizers and harsh topical steroidal treatments, which are not recommended for long term use, nor highly effective. Second line treatment options include nonsteroidal creams which are often powerful immunosuppressants that were designed for transplant patients to reduce immune rejection but therefore can increase the risk of bacterial infections. The next line of treatments is either injectable mABs or oral immunomodulators which are reserved for the most severe cases of atopic dermatitis.

Anti-inflammatory therapeutics for atopic dermatitis such as CIBINQO (abrocitinib) by Pfizer work by inhibiting the Janus kinase (JAKI) via blocking its adenosine triphosphate binding site. Janus kinase activity is thought to contribute to inflammation, so blocking its signaling pathway is thought to reduce inflammation and itchiness and to increase skin clarity. However, this mechanism of action is currently unverified, and the systemic effect of this anti-inflammatory therapeutic is undesirable—it blocks the Janus kinase signaling pathway throughout the body, and is not targeted only to the areas of the body affected by atopic dermatitis.

There is a clear market gap for treatments that address mild to moderate disease, which are non-steroidal, and which possess fewer side effects than currently available treatments, especially in youth. There is also an urgent need for treatments that are designed to work locally to treat mild flare-ups and to provide comfort to the patients at the time of administration. Given the common occurrence in young patients, there is also a need to avoid the use of powerful immunosuppressants.

Intact collagen generally includes anti-inflammatory sites such as LAIR-1. LAIR-1 can act as a check point inhibitor by reducing the inflammatory cytokine and T-Cell activation. In inflammatory disorders such as atopic dermatitis/eczema, high T-Cell and immune activation is often coupled with an increase of MMPs. MMPs decrease barrier function by cleaving collagen. Thus, there is a reduction in natural collagen-based checkpoint inhibitors such as LAIR-1. This increase of MMPs also brings an abundance of damaged collagen for CHPs to bind to. In order to downregulate the immune response related to T-cell mediated conditions typically present within the skin as a result of atopic dermatitis/eczema and to avoid use of systemic treatments and powerful immunosuppressants, bioACTIVE CHPs can increase the number anti-inflammatory sites such as LAIR-1. By using the anti-inflammatory bioACTIVE CHPs as set forth herein in conjunction with other types of bioACTIVE CHPs designed to increase cell migration to the affected areas, keep resident cells in the damaged region, and/or increase the collagen production, CHPs can restore barrier function and down regulate the T-Cell mediated inflammatory pathway, creating fast relief and enhanced healing.

### Anti-Inflammatory, Bioactive Collagen Hybridizing Peptides (CHPs)

The present disclosure relates to anti-inflammatory bioACTIVE collagen hybridizing peptides (CHPs), compositions including CHPs, and methods of using the CHPs and compositions described herein. The collagen hybridizing peptide (CHP) is a short repeating Gly-X-Y tripeptide that is capable of specifically binding to degraded collagen with no affinity for intact collagen molecules. CHPs can distinguish damaged collagen by recognizing the structural motif of the poly-proline II helix of individual alpha chains which is not available on intact collagen molecules, and CHPs can then re-form the collagen triple helix through hybridization with the damaged collagen, forming a highly stable and long-lasting bond. The present disclosure relates to novel bioACTIVE versions of CHPs that can be used to localize collagen based anti-inflammatory sequences to directly downregulate the inflammatory responses at sites with damaged collagen, thereby promoting collagen repair and mitigating further inflammatory damage.

The present disclosure further relates to methods of administering CHPs and compositions including the CHPs to downregulate inflammatory response, and to treat, reduce, prevent, or inhibit disorders, conditions, or diseases involving excessive inflammation, or to enhance other treatments for such disorders, conditions, or diseases. Conditions and treatments including, but not limited to, dry-eye disease (Xeropthalmia), corneal abrasion, diabetic retinopathy, atopic dermatitis (i.e., eczema), psoriasis, vitiligo, bullous pemphigoid, pemphigus vulgarus, Steven's Johnson's syndrome, toxic epidermal necrolysis, osteoarthritis, rheumatoid arthritis, psoriatic arthritis diabetic wounds, pressure ulcerations, wound healing, chemical and other burns, Crohn's Disease, ulcerative colitis, ankylosing spondylitis, eosinophilic esophagitis, primary biliary cholangitis, ascending cholangitis, celiac disease, chronic UTI, solid tumor micro environment signaling, next generation topical antibiotics, granulomatosis with polyangiitis, eosinophilic granulomatosis, Goodpasture syndrome, multiple myeloma, scleroderma, CREST syndrome, IPF, dermatitis herpetaformins, keloid, takayatsus, giant cell arteritis, and adhesive capsulitis may all be treated, enhanced, reduced, prevented, or inhibited according to methods disclosed herein. The methods also provide for increasing immune checkpoints in a subject via administration of the CHPs and compositions disclosed herein. Furthermore, methods for downregulating immune-related cytokines, downregulating immune cells, and preventing differentiation of immune cells are also provided.

In one aspect, the present disclosure provides a collagen hybridizing peptide (CHP) including a sequence represented by Formula I: Ac—S-(Gly-X-Y)ₙ-bioactive-(Gly-X-Y)ₘ, wherein Ac is an acetyl capping group; S is zero or more spacer molecules; X is proline or modified proline; Gly is glycine; Y is hydroxyproline or any other amino acid; n is a number from 1 to 20; m is a number from 1-20. The bioactive may be an amino acid sequence having from 3 to 27 amino acid residues. The bioactive may have bioactivity. The bioactive may comprise at least one sequence selected from the group consisting of a leukocyte-associated immunoglobulin-like receptor sequence (e.g., one or both of a LAIR-1 and LAIR-2 sequence), a G6b-B recognition site sequence, osteoclast-associated receptor (OSCAR) sequence, a glycoprotein VI (GPVI) sequence, and a FcR-γ domain sequence.

In an embodiment of the CHP disclosed herein, X is proline. In another embodiment of the CHP disclosed herein, X is a modified proline. In some embodiments, the modified proline is 2S,4S-4-fluoroproline, (flp) cis-conformation.

In an embodiment of the CHP disclosed herein, Y is hydroxyproline. In another embodiment of the CHP disclosed herein, Y is an amino acid. In some embodiments, the amino acid is 2S,4R-4-fluoroproline, (Flp) trans-conformation.

In an embodiment of the CHP disclosed herein, n is 1. In another embodiment of the CHP disclosed herein, n is 2. In another embodiment of the CHP disclosed herein, n is 3. In another embodiment of the CHP disclosed herein, n is 4. In another embodiment of the CHP disclosed herein, n is 5. In another embodiment of the CHP disclosed herein, n is 6. In another embodiment of the CHP disclosed herein, n is 7. In another embodiment of the CHP disclosed herein, n is 8. In another embodiment of the CHP disclosed herein, n is 9. In another embodiment of the CHP disclosed herein, n is 10. In another embodiment of the CHP disclosed herein, n is 11. In another embodiment of the CHP disclosed herein, n is 12. In another embodiment of the CHP disclosed herein, n is 13. In another embodiment of the CHP disclosed herein, n is 14. In another embodiment of the CHP disclosed herein, n is 15. In another embodiment of the CHP disclosed herein, n is 16. In another embodiment of the CHP disclosed herein, n is 17. In another embodiment of the CHP disclosed herein, n is 18. In another embodiment of the CHP disclosed herein, n is 19. In another embodiment of the CHP disclosed herein, n is 20.

In an embodiment of the CHP disclosed herein, m is 1. In another embodiment of the CHP disclosed herein, m is 2. In another embodiment of the CHP disclosed herein, m is 3. In another embodiment of the CHP disclosed herein, m is 4. In another embodiment of the CHP disclosed herein, m is 5. In another embodiment of the CHP disclosed herein, m is 6. In another embodiment of the CHP disclosed herein, m is 7. In another embodiment of the CHP disclosed herein, m is 8. In another embodiment of the CHP disclosed herein, m is 9. In another embodiment of the CHP disclosed herein, m is 10. In another embodiment of the CHP disclosed herein, m is 11. In another embodiment of the CHP disclosed herein, m is 12. In another embodiment of the CHP disclosed herein, m is 13. In another embodiment of the CHP disclosed herein, m is 14. In another embodiment of the CHP disclosed herein, m is 15. In another embodiment of the CHP disclosed herein, m is 16. In another embodiment of the CHP disclosed herein, mis 17. In another embodiment of the CHP disclosed herein, m is 18. In another embodiment of the CHP disclosed herein, m is 19. In another embodiment of the CHP disclosed herein, m is 20.

In an embodiment of the CHP disclosed herein, the bioactive has bioactivity. In a further embodiment of the CHP disclosed herein, the bioactive is an amino acid sequence having 3 amino acid residues. In a further embodiment of the CHP disclosed herein, the bioactive is an amino acid sequence having 4 amino acid residues. In a further embodiment of the CHP disclosed herein, the bioactive is an amino acid sequence having 5 amino acid residues. In a further embodiment of the CHP disclosed herein, the bioactive is an amino acid sequence having 6 amino acid residues. In a further embodiment of the CHP disclosed herein, the bioactive is an amino acid sequence having 7 amino acid residues. In a further embodiment of the CHP disclosed herein, the bioactive is an amino acid sequence having 8 amino acid residues. In a further embodiment of the CHP disclosed herein, the bioactive is an amino acid sequence having 9 amino acid residues. In a further embodiment of the CHP disclosed herein, the bioactive is an amino acid sequence having 10 amino acid residues. In a further embodiment of the CHP disclosed herein, the bioactive is an amino acid sequence having 11 amino acid residues. In a further embodiment of the CHP disclosed herein, the bioactive is an amino acid sequence having 12 amino acid residues. In a further embodiment of the CHP disclosed herein, the bioactive is an amino acid sequence having 13 amino acid residues. In a further embodiment of the CHP disclosed herein, the bioactive is an amino acid sequence having 14 amino acid residues. In a further embodiment of the CHP disclosed herein, the bioactive is an amino acid sequence having 15 amino acid residues. In a further embodiment of the CHP disclosed herein, the bioactive is an amino acid sequence having 16 amino acid residues. In a further embodiment of the CHP disclosed herein, the bioactive is an amino acid sequence having 17 amino acid residues. In a further embodiment of the CHP disclosed herein, the bioactive is an amino acid sequence having 18 amino acid residues. In a further embodiment of the CHP disclosed herein, the bioactive is an amino acid sequence having 19 amino acid residues. In a further embodiment of the CHP disclosed herein, the bioactive is an amino acid sequence having 20 amino acid residues. In a further embodiment of the CHP disclosed herein, the bioactive is an amino acid sequence having 21 amino acid residues. In a further embodiment of the CHP disclosed herein, the bioactive is an amino acid sequence having 22 amino acid residues. In a further embodiment of the CHP disclosed herein, the bioactive is an amino acid sequence having 23 amino acid residues. In a further embodiment of the CHP disclosed herein, the bioactive is an amino acid sequence having 24 amino acid residues. In a further embodiment of the CHP disclosed herein, the bioactive is an amino acid sequence having 25 amino acid residues. In a further embodiment of the CHP disclosed herein, the bioactive is an amino acid sequence having 26 amino acid residues. In a further embodiment of the CHP disclosed herein, the bioactive is an amino acid sequence having 27 amino acid residues.

In an embodiment of the CHP disclosed herein, the bioactive may comprise sequences from a leukocyte-associated immunoglobulin-like receptor sequence (e.g., one or both of a LAIR-1 and LAIR-2 sequence), a G6b-B recognition site sequence, an osteoclast-associated receptor (OSCAR) domain sequence, a glycoprotein VI (GPVI) sequence, and a FcR-γ domain sequence. In a further embodiment of the CHP disclosed herein, the bioactive comprises a LAIR-1 sequence. In a further embodiment of the CHP disclosed herein, the bioactive comprises a LAIR-2 sequence. In a further embodiment of the CHP disclosed herein, the bioactive comprises a G6b-B recognition site sequence. In a further embodiment of the CHP disclosed herein, the bioactive comprises an osteoclast-associated receptor (OSCAR) domain sequence. In a further embodiment of the CHP disclosed herein, the bioactive comprises a glycoprotein VI (GPVI) sequence. In a further embodiment of the CHP disclosed herein, the bioactive comprises a FcR-γ domain sequence.

In an embodiment of the CHP disclosed herein, the CHP comprises the sequence of any one of SEQ ID NOs: 1-64 as shown in Table 1 below.

TABLE 1

| SEQUENCE | |
|---|---|
| ID | CHP AMINO ACID SEQUENCE |
| SEQ. ID 1 | Ac-(GPP)ₓ-OGQDGLAGPK-(GPP)_{y} |
| SEQ. ID 2 | Ac-(GPO)ₓ-GQDGLAGPK-(GPO)_{y} |
| SEQ. ID 3 | Ac-(GfO)ₓ-GQDGLAGPK-(GfO)_{y} |
| SEQ. ID 4 | Ac-(GPP)ₓ-OGQDGLAGPK-(GPO)_{y} |
| SEQ. ID 5 | Ac-(GPO)ₓ-GQDGLAGPK-(GPP)_{y} |
| SEQ. ID 6 | Ac-(GPP)ₓ-GQDGLAGPK-(GfO)_{y} |
| SEQ. ID 7 | Ac-(GPO)ₓ-GQDGLAGPK-(GfO)_{y} |
| SEQ. ID 8 | Ac-(GfO)ₓ-GQDGLAGPK-(GPO)_{y} |
| SEQ. ID 9 | Ac-(GfO)ₓ-GQDGLAGPK-(GPP)_{y} |
| SEQ. ID 10 | Ac-(GPP)ₓ-VTYAQL-(GPP)_{y} |
| SEQ. ID 11 | Ac-(GPP)ₓ-VTYAQL-(GPO)_{y} |
| SEQ. ID 12 | Ac-(GPP)ₓ-VTYAQL-(GfO)_{y} |
| SEQ. ID 13 | Ac-(GPO)ₓ-VTYAQL-(GPO)_{y} |
| SEQ. ID 14 | Ac-(GPO)ₓ-VTYAQL-(GPP)_{y} |
| SEQ. ID 15 | Ac-(GPO)ₓ-VTYAQL-(GfO)_{y} |
| SEQ. ID 16 | Ac-(GfO)ₓ-VTYAQL-(GfO)_{y} |
| SEQ. ID 17 | Ac-(GfO)ₓ-VTYAQL-(GPO)_{y} |
| SEQ. ID 18 | Ac-(GfO)ₓ-VTYAQL-(GPP)_{y} |
| SEQ. ID 19 | Ac-(GPP)ₓ-ITYAAV-(GPP)_{y} |
| SEQ. ID 20 | Ac-(GPP)ₓ-ITYAAV-(GPO)_{y} |
| SEQ. ID 21 | Ac-(GPP)ₓ-ITYAAV-(GfO)_{y} |
| SEQ. ID 22 | Ac-(GPO)ₓ-ITYAAV-(GPO)_{y} |
| SEQ. ID 23 | Ac-(GPO)ₓ-ITYAAV-(GPP)_{y} |
| SEQ. ID 24 | Ac-(GPO)ₓ-ITYAAV-(GfO)_{y} |
| SEQ. ID 25 | Ac-(GfO)ₓ-ITYAAV-(GfO)_{y} |
| SEQ. ID 26 | Ac-(GfO)ₓ-ITYAAV-(GPP)_{y} |
| SEQ. ID 27 | Ac-(GfO)ₓ-ITYAAV-(GPO)_{y} |
| SEQ. ID 28 | Ac-(GPP)ₓ-LLYADL-(GPP)_{y} |
| SEQ. ID 29 | Ac-(GPP)ₓ-LLYADL-(GPO)_{y} |
| SEQ. ID 30 | Ac-(GPP)ₓ-LLYADL-(GfO)_{y} |
| SEQ. ID 31 | Ac-(GPO)ₓ-LLYADL-(GPO)_{y} |
| SEQ. ID 32 | Ac-(GPO)ₓ-LLYADL-(GPP)_{y} |
| SEQ. ID 33 | Ac-(GPO)ₓ-LLYADL-(GfO)_{y} |
| SEQ. ID 34 | Ac-(GfO)ₓ-LLYADL-(GfO)_{y} |
| SEQ. ID 35 | Ac-(GfO)ₓ-LLYADL-(GPP)_{y} |
| SEQ. ID 36 | Ac-(GfO)ₓ-LLYADL-(GPO)_{y} |
| SEQ. ID 37 | Ac-(GfO)ₓ-LLYADL-(GfO)_{y} |
| SEQ. ID 38 | Ac-(GPP)ₓ-TIYAVV-(GPP)_{y} |
| SEQ. ID 39 | Ac-(GPP)ₓ-TIYAVV-(GPO)_{y} |
| SEQ. ID 40 | Ac-(GPP)ₓ-TIYAVV-(GfO)_{y} |
| SEQ. ID 41 | Ac-(GPO)ₓ-TIYAVV-(GPO)_{y} |
| SEQ. ID 42 | Ac-(GPO)ₓ-TIYAVV-(GPP)_{y} |
| SEQ. ID 43 | Ac-(GPO)ₓ-TIYAVV-(GfO)_{y} |
| SEQ. ID 44 | Ac-(GfO)ₓ-TIYAVV-(GfO)_{y} |
| SEQ. ID 45 | Ac-(GfO)ₓ-TIYAVV-(GPP)_{y} |
| SEQ. ID 46 | Ac-(GfO)ₓ-TIYAVV-(GPO)_{y} |
| SEQ. ID 47 | Ac-(GPP)ₓ-ITYSLL-(GPP)_{y} |
| SEQ. ID 48 | Ac-(GPP)ₓ-ITYSLL-(GPO)_{y} |
| SEQ. ID 49 | Ac-(GPP)ₓ-ITYSLL-(GfO)_{y} |
| SEQ. ID 50 | Ac-(GPO)ₓ-ITYSLL-(GPO)_{y} |
| SEQ. ID 51 | Ac-(GPO)ₓ-ITYSLL-(GPP)_{y} |
| SEQ. ID 52 | Ac-(GPO)ₓ-ITYSLL-(GfO)_{y} |
| SEQ. ID 53 | Ac-(GfO)ₓ-ITYSLL-(GfO)_{y} |
| SEQ. ID 54 | Ac-(GfO)ₓ-ITYSLL-(GPP)_{y} |
| SEQ. ID 55 | Ac-(GfO)ₓ-ITYSLL-(GPO)_{y} |
| SEQ. ID 56 | Ac-(GPP)ₓ-GAOGLRGGAGPOGPEGGKGAAGPOGPO- |
| | (GPP)_{y} |
| SEQ. ID 57 | Ac-(GPP)ₓ-GAOGLRGGAGPOGPEGGKGAAGPOGPO- |
| | (GPO)_{y} |
| SEQ. ID 58 | Ac-(GPP)ₓ-GAOGLRGGAGPOGPEGGKGAAGPOGPO- |
| | (GfO)_{y} |
| SEQ. ID 59 | Ac-(GPO)ₓ-GAOGLRGGAGPOGPEGGKGAAGPOGPO- |
| | (GPO)_{y} |
| SEQ. ID 60 | Ac-(GPO)ₓ-GAOGLRGGAGPOGPEGGKGAAGPOGPO- |
| | (GPP)_{y} |
| SEQ. ID 61 | Ac-(GPO)ₓ-GAOGLRGGAGPOGPEGGKGAAGPOGPO- |
| | (GfO)_{y} |
| SEQ. ID 62 | Ac-(GfO)ₓ-GAOGLRGGAGPOGPEGGKGAAGPOGPO- |
| | (GfO)_{y} |
| SEQ. ID 63 | Ac-(GfO)ₓ-GAOGLRGGAGPOGPEGGKGAAGPOGPO- |
| | (GPO)_{y} |
| SEQ. ID 64 | Ac-(GfO)ₓ-GAOGLRGGAGPOGPEGGKGAAGPOGPO- |
| | (GPP)_{y} |
| SEQ. ID 65 | Ac-(GPP)ₓ-OGPOGP-(GPP)_{y} |
| SEQ. ID 66 | Ac-(GPP)ₓ-OGPOGP-(GPO)_{y} |
| SEQ. ID 67 | Ac-(GPP)ₓ-OGPOGP-(GfO)_{y} |
| SEQ. ID 68 | Ac-(GPO)ₓ-OGPOGP-(GPO)_{y} |
| SEQ. ID 69 | Ac-(GPO)ₓ-OGPOGP-(GPP)_{y} |
| SEQ. ID 70 | Ac-(GPO)ₓ-OGPOGP-(GfO)_{y} |
| SEQ. ID 71 | Ac-(GfO)ₓ-OGPOGP-(GfO)_{y} |
| SEQ. ID 72 | Ac-(GfO)ₓ-OGPOGP-(GPO)_{y} |
| SEQ. ID 73 | Ac-(GfO)ₓ-OGPOGP-(GPP)_{y} |
| SEQ. ID 74 | Ac-(GPP)ₓ-AGPOGP-(GPP)_{y} |
| SEQ. ID 75 | Ac-(GPP)ₓ-AGPOGP-(GPO)_{y} |
| Seq ID 76 | Ac-(GPP)ₓ-AGPOGP-(GfO)_{y} |
| Seq ID 77 | Ac-(GPO)ₓ-AGPOGP-(GPO)_{y} |
| Seq ID 78 | Ac-(GPO)ₓ-AGPOGP-(GPP)_{y} |
| Seq ID 79 | Ac-(GPO)ₓ-AGPOGP-(GfO)_{y} |
| Seq ID 80 | Ac-(GfO)ₓ-AGPOGP-(GfO)_{y} |
| Seq ID 81 | Ac-(GfO)ₓ-AGPOGP-(GPO)_{y} |
| Seq ID 82 | Ac-(GfO)ₓ-AGPOGP-(GPP)_{y} |
| Seq ID 83 | Ac-(GPO)5 |
| Seq ID 84 | Ac-(GPP)ₓ-GPOGPAGFO-(GPP)_{y} |
| Seq ID 85 | Ac-(GPP)ₓ-GPOGPAGFO-(GPO)_{y} |
| Seq ID 86 | Ac-(GPP)ₓ-GPOGPAGFO-(GfO)_{y} |
| Seq ID 87 | Ac-(GPO)ₓ-GPOGPAGFO-(GPO)_{y} |
| Seq ID 88 | Ac-(GPO)ₓ-GPOGPAGFO-(GPP)_{y} |
| Seq ID 89 | Ac-(GPO)ₓ-GPOGPAGFO-(GfO)_{y} |
| Seq ID 90 | Ac-(GfO)ₓ-GPOGPAGFO-(GfO)_{y} |
| Seq ID 91 | Ac-(GfO)ₓ-GPOGPAGFO-(GPO)_{y} |
| Seq ID 92 | Ac-(GfO)ₓ-GPOGPAGFO-(GPP)_{y} |
| Seq ID 93 | VTYAQL |
| Seq ID 94 | ITYAAV |
| Seq ID 95 | LLYADL |
| Seq ID 96 | TIYAVV |
| Seq ID 97 | ITYSLL |
| Seq ID 98 | GAOGLRGGAGPOGPEGGKGAAGPOGPO |
| Seq ID 99 | GQDGLAGPK |
| Seq ID 100 | OGQDGLAGPK |
| Seq ID 101 | OGPOGP |
| Seq ID 102 | AGPOGP |
| Seq ID 103 | GPOGPOGPOGPOGPO |
| Seq ID 104 | GPOGPAGFO |
| Seq ID 105 | (GGG)_{z} |
| Seq ID 106 | Biotin-GGG-(GPO)₉ |

The Ac- is an acetyl (capping) group at the N-terminus, the G- is Glycine, the P- is proline, the O- is hydroxyproline, the f- is 2S,4S-4-fluoroproline, x is any integer from 1-7, y is any integer from 1-7, and z is any integer from 1-5.

In some embodiments, the Acetyl group (Ac) attached to the N-Terminus of the CHPs as disclosed herein is replaced with one or more of a therapeutic drug, a fluorescent dye, and other imaging molecule or modality. Therapeutic drugs that replace the Acetyl group at the N-terminus are not a part of the bioactive amino acid sequence in the middle of the CHP. The N-terminus modifications of such embodiments do not inhibit or interfere with the bioactive amino acid sequences of the CHP, and they do not prevent or inhibit the CHP from binding to damaged collagen.

In a further embodiment of the CHP disclosed herein, the CHP comprises SEQ ID NO: 1. In a further embodiment of the CHP disclosed herein, the CHP comprises SEQ ID NO: 2. In a further embodiment of the CHP disclosed herein, the CHP comprises SEQ ID NO: 3. In a further embodiment of the CHP disclosed herein, the CHP comprises SEQ ID NO: 4. In a further embodiment of the CHP disclosed herein, the CHP comprises SEQ ID NO: 5. In a further embodiment of the CHP disclosed herein, the CHP comprises SEQ ID NO: 6. In a further embodiment of the CHP disclosed herein, the CHP comprises SEQ ID NO: 7. In a further embodiment of the CHP disclosed herein, the CHP comprises SEQ ID NO: 8. In a further embodiment of the CHP disclosed herein, the CHP comprises SEQ ID NO: 9. In a further embodiment of the CHP disclosed herein, the CHP comprises SEQ ID NO: 10. In a further embodiment of the CHP disclosed herein, the CHP comprises SEQ ID NO: 11. In a further embodiment of the CHP disclosed herein, the CHP comprises SEQ ID NO: 12. In a further embodiment of the CHP disclosed herein, the CHP comprises SEQ ID NO: 13. In a further embodiment of the CHP disclosed herein, the CHP comprises SEQ ID NO: 14. In a further embodiment of the CHP disclosed herein, the CHP comprises SEQ ID NO: 15. In a further embodiment of the CHP disclosed herein, the CHP comprises SEQ ID NO: 16. In a further embodiment of the CHP disclosed herein, the CHP comprises SEQ ID NO: 17. In a further embodiment of the CHP disclosed herein, the CHP comprises SEQ ID NO: 18. In a further embodiment of the CHP disclosed herein, the CHP comprises SEQ ID NO: 19. In a further embodiment of the CHP disclosed herein, the CHP comprises SEQ ID NO: 20. In a further embodiment of the CHP disclosed herein, the CHP comprises SEQ ID NO: 21. In a further embodiment of the CHP disclosed herein, the CHP comprises SEQ ID NO: 22. In a further embodiment of the CHP disclosed herein, the CHP comprises SEQ ID NO: 23. In a further embodiment of the CHP disclosed herein, the CHP comprises SEQ ID NO: 24. In a further embodiment of the CHP disclosed herein, the CHP comprises SEQ ID NO: 25. In a further embodiment of the CHP disclosed herein, the CHP comprises SEQ ID NO: 26. In a further embodiment of the CHP disclosed herein, the CHP comprises SEQ ID NO: 27. In a further embodiment of the CHP disclosed herein, the CHP comprises SEQ ID NO: 28. In a further embodiment of the CHP disclosed herein, the CHP comprises SEQ ID NO: 29. In a further embodiment of the CHP disclosed herein, the CHP comprises SEQ ID NO: 30. In a further embodiment of the CHP disclosed herein, the CHP comprises SEQ ID NO: 31. In a further embodiment of the CHP disclosed herein, the CHP comprises SEQ ID NO: 32. In a further embodiment of the CHP disclosed herein, the CHP comprises SEQ ID NO: 33. In a further embodiment of the CHP disclosed herein, the CHP comprises SEQ ID NO: 34. In a further embodiment of the CHP disclosed herein, the CHP comprises SEQ ID NO: 35. In a further embodiment of the CHP disclosed herein, the CHP comprises SEQ ID NO: 36. In a further embodiment of the CHP disclosed herein, the CHP comprises SEQ ID NO: 37. In a further embodiment of the CHP disclosed herein, the CHP comprises SEQ ID NO: 38. In a further embodiment of the CHP disclosed herein, the CHP comprises SEQ ID NO: 39. In a further embodiment of the CHP disclosed herein, the CHP comprises SEQ ID NO: 40. In a further embodiment of the CHP disclosed herein, the CHP comprises SEQ ID NO: 41. In a further embodiment of the CHP disclosed herein, the CHP comprises SEQ ID NO: 42. In a further embodiment of the CHP disclosed herein, the CHP comprises SEQ ID NO: 43. In a further embodiment of the CHP disclosed herein, the CHP comprises SEQ ID NO: 44. In a further embodiment of the CHP disclosed herein, the CHP comprises SEQ ID NO: 45. In a further embodiment of the CHP disclosed herein, the CHP comprises SEQ ID NO: 46. In a further embodiment of the CHP disclosed herein, the CHP comprises SEQ ID NO: 47. In a further embodiment of the CHP disclosed herein, the CHP comprises SEQ ID NO: 48. In a further embodiment of the CHP disclosed herein, the CHP comprises SEQ ID NO: 49. In a further embodiment of the CHP disclosed herein, the CHP comprises SEQ ID NO: 50. In a further embodiment of the CHP disclosed herein, the CHP comprises SEQ ID NO: 51. In a further embodiment of the CHP disclosed herein, the CHP comprises SEQ ID NO: 52. In a further embodiment of the CHP disclosed herein, the CHP comprises SEQ ID NO: 53. In a further embodiment of the CHP disclosed herein, the CHP comprises SEQ ID NO: 54. In a further embodiment of the CHP disclosed herein, the CHP comprises SEQ ID NO: 55. In a further embodiment of the CHP disclosed herein, the CHP comprises SEQ ID NO: 56. In a further embodiment of the CHP disclosed herein, the CHP comprises SEQ ID NO: 57. In a further embodiment of the CHP disclosed herein, the CHP comprises SEQ ID NO: 58. In a further embodiment of the CHP disclosed herein, the CHP comprises SEQ ID NO: 59. In a further embodiment of the CHP disclosed herein, the CHP comprises SEQ ID NO: 60. In a further embodiment of the CHP disclosed herein, the CHP comprises SEQ ID NO: 61. In a further embodiment of the CHP disclosed herein, the CHP comprises SEQ ID NO: 62. In a further embodiment of the CHP disclosed herein, the CHP comprises SEQ ID NO: 63. In a further embodiment of the CHP disclosed herein, the CHP comprises SEQ ID NO: 64. In a further embodiment of the CHP disclosed herein, the CHP comprises SEQ ID NO: 65. In a further embodiment of the CHP disclosed herein, the CHP comprises SEQ ID NO: 66. In a further embodiment of the CHP disclosed herein, the CHP comprises SEQ ID NO: 67. In a further embodiment of the CHP disclosed herein, the CHP comprises SEQ ID NO: 68. In a further embodiment of the CHP disclosed herein, the CHP comprises SEQ ID NO: 69. In a further embodiment of the CHP disclosed herein, the CHP comprises SEQ ID NO: 70. In a further embodiment of the CHP disclosed herein, the CHP comprises SEQ ID NO: 71. In a further embodiment of the CHP disclosed herein, the CHP comprises SEQ ID NO: 72. In a further embodiment of the CHP disclosed herein, the CHP comprises SEQ ID NO: 73. In a further embodiment of the CHP disclosed herein, the CHP comprises SEQ ID NO: 74. In a further embodiment of the CHP disclosed herein, the CHP comprises SEQ ID NO: 75. In a further embodiment of the CHP disclosed herein, the CHP comprises SEQ ID NO: 76. In a further embodiment of the CHP disclosed herein, the CHP comprises SEQ ID NO: 77. In a further embodiment of the CHP disclosed herein, the CHP comprises SEQ ID NO: 78. In a further embodiment of the CHP disclosed herein, the CHP comprises SEQ ID NO: 79. In a further embodiment of the CHP disclosed herein, the CHP comprises SEQ ID NO: 80. In a further embodiment of the CHP disclosed herein, the CHP comprises SEQ ID NO: 81. In a further embodiment of the CHP disclosed herein, the CHP comprises SEQ ID NO: 82. In a further embodiment of the CHP disclosed herein, the CHP comprises SEQ ID NO: 83. In a further embodiment of the CHP disclosed herein, the CHP comprises SEQ ID NO: 84. In a further embodiment of the CHP disclosed herein, the CHP comprises SEQ ID NO: 85. In a further embodiment of the CHP disclosed herein, the CHP comprises SEQ ID NO: 86. In a further embodiment of the CHP disclosed herein, the CHP comprises SEQ ID NO: 87. In a further embodiment of the CHP disclosed herein, the CHP comprises SEQ ID NO: 88. In a further embodiment of the CHP disclosed herein, the CHP comprises SEQ ID NO: 89. In a further embodiment of the CHP disclosed herein, the CHP comprises SEQ ID NO: 90. In a further embodiment of the CHP disclosed herein, the CHP comprises SEQ ID NO: 91. In a further embodiment of the CHP disclosed herein, the CHP comprises SEQ ID NO: 92. In a further embodiment of the CHP disclosed herein, the CHP comprises SEQ ID NO: 93. In a further embodiment of the CHP disclosed herein, the CHP comprises SEQ ID NO: 94. In a further embodiment of the CHP disclosed herein, the CHP comprises SEQ ID NO: 95. In a further embodiment of the CHP disclosed herein, the CHP comprises SEQ ID NO: 96. In a further embodiment of the CHP disclosed herein, the CHP comprises SEQ ID NO: 97. In a further embodiment of the CHP disclosed herein, the CHP comprises SEQ ID NO: 98. In a further embodiment of the CHP disclosed herein, the CHP comprises SEQ ID NO: 99. In a further embodiment of the CHP disclosed herein, the CHP comprises SEQ ID NO: 100. In a further embodiment of the CHP disclosed herein, the CHP comprises SEQ ID NO: 101. In a further embodiment of the CHP disclosed herein, the CHP comprises SEQ ID NO: 102. In a further embodiment of the CHP disclosed herein, the CHP comprises SEQ ID NO: 103. In a further embodiment of the CHP disclosed herein, the CHP comprises SEQ ID NO: 104.

In an embodiment of the CHP disclosed herein, the bioactive comprises a LAIR-1 and/or LAIR-2 receptor sequence comprising a sequence having at least 85% sequence identity to any one of SEQ ID NO: 1-27, 56-64, 93, 94, and 98-100.

In an embodiment of the CHP disclosed herein, the bioactive comprises a G6b-B recognition site sequence comprising a sequence having at least 85% sequence identity to any one of SEQ ID NO: 28-46, 95, and 96.

In another embodiment of the CHP disclosed herein, the bioactive comprises a FcR-γ domain sequence comprising a sequence having at least 85% sequence identity to any one of SEQ ID NO: 47-55 and 97.

In another embodiment of the CHP disclosed herein, the bioactive comprises a GPVI domain sequence comprising a sequence having at least 85% sequence identity to any one of SEQ ID NO: 65-83 and 101-103.

In another embodiment of the CHP disclosed herein, the bioactive comprises an OSCAR domain sequence comprising a sequence having at least 85% sequence identity to any one of SEQ ID NO: 84-92 and 104.

In an embodiment of the CHP disclosed herein, the bioactive comprises a sequence selected from the group consisting of SEQ ID NO: 93-104.

In a further embodiment of the CHP disclosed herein, the bioactive comprises SEQ ID NO: 93. In a further embodiment of the CHP disclosed herein, the bioactive comprises SEQ ID NO: 94. In a further embodiment of the CHP disclosed herein, the bioactive comprises SEQ ID NO: 95. In a further embodiment of the CHP disclosed herein, the bioactive comprises SEQ ID NO: 96. In a further embodiment of the CHP disclosed herein, the bioactive comprises SEQ ID NO: 97. In a further embodiment of the CHP disclosed herein, the bioactive comprises SEQ ID NO: 98. In a further embodiment of the CHP disclosed herein, the bioactive comprises SEQ ID NO: 99. In a further embodiment of the CHP disclosed herein, the bioactive comprises SEQ ID NO: 100. In a further embodiment of the CHP disclosed herein, the CHP comprises SEQ ID NO: 101. In a further embodiment of the CHP disclosed herein, the CHP comprises SEQ ID NO: 102. In a further embodiment of the CHP disclosed herein, the CHP comprises SEQ ID NO: 103. In a further embodiment of the CHP disclosed herein, the CHP comprises SEQ ID NO: 104.

In an embodiment of the CHP disclosed herein, S is at least 1 spacer molecule. In a further embodiment, S comprises a number of spacer molecules greater than 0. In some embodiments, the spacer molecule is a linear PEG molecule, wherein the linear PEG molecule can have an unlimited number of PEG repeats. In some embodiments, the spacer molecule is an aminohexanoic acid. In other embodiments, the spacer molecule is an unlimited number of aminohexanoic acids connected sequentially. In other embodiments, the spacer molecule is one or more amino acids.

In an embodiment of the CHP as disclosed herein, a first bioactive CHP is bound to two other bioactive CHPs to form a trimeric structure presenting a trimeric cell-binding site, wherein the first bioactive CHP extends beyond the trimeric cell-binding site as a monomeric “tail” section. The monomeric “tail” section of the first CHP localizes to the damaged collagen via structural recognition of collagen individual alpha chains, while the trimeric cell-binding site comprising the first CHP bound to two other CHPs presents bioactive sites in a triple helical format for ease of binding with cell receptors. In some instances, a monomeric bioACTIVE CHP may not be available or “visible” to immune cells depending on its 3D orientation within the tissue, or within the damaged collagen strands that form a triple helix with the monomeric bioACTIVE CHP. This may cause a weaker binding affinity of the bioactive sequence with the applicable cell receptor, thereby reducing the effectiveness of the monomeric bioactive CHP. Thus, a first bioactive CHP bound to two other bioactive CHPs to form a trimeric structure presenting cell receptor binding sites in a triple-helical conformation (see FIG. 3) may help address this concern. The bioactive sequence binding site will be available regardless of the 3D orientation or CHP binding location on damaged collagen; thus, high affinity binding sites will be available for cell interaction. The monomeric “tail” section of the first bioactive CHP ensures the CHP can localize and bind to damaged collagen in a wound/lesion by maintaining the ability to recognize exposed collagen alpha chains and to fold with denatured collagen strands.

In an embodiment of the CHP disclosed herein, the design of the triple-helical cell binding site with a monomeric CHP “tail” can also include creating the triple-helical cell binding site using three bioACTIVE CHPs and then allowing the three bioACTIVE CHPs to fold together, before attaching the monomeric “tail” to either the N-terminus or the C-terminus of one of the three bioACTIVE CHP sequences in the already-formed triple-helix via conjugation chemistry. The three bioACTIVE CHPs in the triple-helical formation have the same amino acid sequence.

In one embodiment, to attach the monomeric “tail” to the C-terminus of the triple-helical cell binding site, two bioACTIVE CHPs having the same amino acid sequence are synthesized using standard Fmoc-mediated SPPS. A single bioACTIVE sequence (having the same amino acid sequence and cell binding site as two the Fmoc-mediated SPPS-synthesized bioACTIVE CHPs) will be synthesized using Fmoc-mediated SPPS but using Wang Resin instead of Rink-amide resin. This will result in a free carboxylic acid (—COOH) on the C-terminus of the single bioACTIVE CHP sequence produced using Wang Resin upon cleavage from the resin. Then, the bioACTIVE CHPs are mixed at a 2:1 ratio (bioACTIVE sequence without —COOH: bioACTIVE with —COOH, respectively), and allowed to fold at 4° C. for 72 hrs to become a triple helix. Next, the monomeric CHP “tail,” which does not contain any bioACTIVE sequence cell binding sites, is synthesized separately using rink-amide resin and following standard Fmoc-mediated SPPS procedures. The N-Terminal of the monomeric CHP “tail” remains uncapped, with a primary amine (NH₂—) group available for binding. The monomeric “tail” CHP is then attached via solution-phase coupling using activating reagents which allow for amide bond formation between the —COOH of the single bioACTIVE CHP sequence produced using Wang Resin in the triple helical cell binding site, and the free amine groups (NH₂—) on the N-terminal of the monomeric “tail” CHP. Once the monomeric CHP “Tail” is attached, the bioACTIVE CHP is purified using dialysis with a MWCO of 3000-5000 Da to remove any unbound CHPs.

In yet a further embodiment, to attach the monomeric CHP “tail” to the N-terminal, the monomeric “Tail” CHP is synthesized on the Wang Resin to produce a reactive-COOH group on its C-Terminal side. To form the triple-helical cell binding site, three bioACTIVE CHP sequences are synthesized on Rink-Amide Resin, and one of the three bioACTIVE CHPs is left uncapped (no Acetyl group) to leave a free amine (NH₂—) on the N-Terminal. The bioACTIVE CHP sequences are then mixed at 2:1 ratio (Acetyl capped bioACTIVE CHP:uncapped bioACTIVE CHP, respectively), and allowed to fold at 4° C. for 72 hours to become a triple helix. Lastly, the monomeric “Tail” CHP is mixed with the already-folded triple-helical cell binding site in solution phase with the correct reagents, and the NH₂ and COOH groups are allowed to form an amide bond through a condensation reaction. In both cases, whichever CHP has the carboxylic acid group needs to be added in excess of the amine group during the peptide bond formation. Once the monomeric CHP “Tail” is attached, the bioACTIVE CHP are purified using dialysis with a MWCO of 3000-5000 Da to remove any unbound CHPs.

In still further embodiments, the monomeric “Tail” CHP is attached using copper-free click chemistry. First, three bioACTIVE CHPs are synthesized; all three have the same amino acid sequence. Two of the bioACTIVE CHP sequences are identical and contain an Acetyl group capping the N-Terminal, while one bioACTIVE CHP has a Dibenzocyclooctyne (DBCO) group attached to the N-terminal instead of an acetyl group capping. This is accomplished by reacting a commercially available DBCO (e.g., DBCO-PEG4-acid, amongst many other commercially available forms of DBCO groups that have different linker sizes or conjugation chemistries) which reacts with the free amine (NH₂—) group on the N-terminal of the uncapped bioACTIVE CHP. The bioACTIVE CHP sequences are then mixed at a 2:1 ratio (bioACTIVE with Acetyl:bioACTIVE with DBCO) and allowed to fold at 4° C. for 72 hrs to become a triple helix. The monomeric “Tail” CHP is synthesized on Wang Resin without a bioACTIVE cell binding sequence so that the C-terminal has a reactive carboxylic acid group (—COOH) to react with an azide group, such as azidoacetic-acid NHS-ester (there are many commercially available forms of azido groups that have different linker sizes or conjugation chemistries which can be used). Once attached, the DBCO group on the N-terminal of the triple-helical cell binding site will specifically bind to the azide group on the C-terminal of the monomeric “Tail” CHP. Once the monomeric CHP “Tail” is attached, the bioACTIVE CHP is purified using dialysis with a MWCO of 3000-5000 Da to remove any unbound CHPs.

In yet another embodiment, to attach the monomeric “Tail” CHP on the C-terminal of the triple-helical cell binding site, the DCBO/azide protocol is followed as set forth above, but the location of the DBCO and azide groups are switched (i.e., the DBCO group is attached to the N-terminal of the monomeric “tail” CHP sequence, while the azide group is attached to the C-terminal of one of the three bioACTIVE CHP sequences of the triple helical formation). Once the monomeric CHP “Tail” is attached, the bioACTIVE CHP is purified using dialysis with a MWCO of 3000-5000 Da to remove any unbound CHPs.

In an aspect, a composition comprising a CHP as disclosed herein is provided. In an embodiment of the composition herein, the composition further comprises a carrier. In a further embodiment of the composition herein, the CHP does not form a triple helix with other CHPs.

In an embodiment of the composition comprising a CHP as disclosed herein, the composition is a cosmetic and/or dermal therapeutic composition. In a further embodiment of the composition, the composition comprises a carrier. In still further embodiments of the composition, the carrier comprises at least one selected from the group consisting of micelles, dendrites, lipids, microemulsions, nanoemulsions, solid lipid nanoparticles, polymers, gels, lens, surfactants, cyclodextrins, inserts, nanostructured lipid carriers, liposomes, transfersomes, ethosomes, niosomes, collagen matrix, extracellular matrix, and artificial extracellular matrix.

In an embodiment of the composition comprising a CHP as disclosed herein, the composition is a CHP bound directly to a collagen matrix mesh or sponge.

In an embodiment of the composition comprising a CHP as disclosed herein, the composition is a topical cream, a saline solution, a gel, a polymer, or a solution for systemic injection within a subject. In a further embodiment, the composition is a topical cream. In a further embodiment, the composition is a saline solution. In a further embodiment, the composition is a gel. In a further embodiment, the composition is a polymer. In a further embodiment, the composition is a solution for systemic injection within a subject.

In one aspect, a method of reducing, preventing, or treating inflammation in a subject is provided, the method comprising administering a CHP as disclosed herein or a composition as disclosed herein to a subject. In an embodiment, the method reduces inflammation in a subject. In an embodiment, the method prevents inflammation in a subject. In another embodiment, the method treats inflammation in a subject.

In an embodiment of the method of reducing, preventing, or treating inflammation in a subject disclosed herein, the step of administering is performed by injection. In an embodiment of the method of reducing, preventing, or treating inflammation in a subject disclosed herein, the step of administering is performed by micro-dermal injection. In an embodiment of the method of reducing, preventing, or treating inflammation in a subject disclosed herein, the step of administering is performed by topical application.

In one aspect, a method of inhibiting or reducing an inflammatory response in a subject is provided, the method comprising administering a CHP as disclosed herein or a composition as disclosed herein to a subject. In an embodiment, the method inhibits an inflammatory response in a subject. In an embodiment, the method reduces an inflammatory response in a subject. In an embodiment of the method disclosed herein, the step of administering is performed by injection. In an embodiment of the method disclosed herein, the step of administering is performed by micro-dermal injection. In an embodiment of the method disclosed herein, the step of administering is performed by topical application.

In another aspect, a method of downregulating immune-related cytokines comprises administering a CHP as disclosed herein or a composition as disclosed herein to a subject. In an embodiment, the immune-related cytokines include, but are not limited to, toll-like receptors (TLR) ligands, interleukins, IFN-α, TLR-9, TNF-α, IL-1β, IL-6, CCL3, IL-16, and/or IL-18.

In further embodiments, the method downregulates one or more TLR ligands. In still further embodiments, the method downregulates TLR-9.

In further embodiments, the method downregulates IFN-α. In further embodiments, the method downregulates CCL3.

In further embodiments, the method downregulates interleukins. In still further embodiments, the method downregulates IL-1β. In still further embodiments, the method downregulates IL-6. In still further embodiments, the method downregulates IL-16. In still further embodiments, the method downregulates IL-18.

In another aspect, a method of preventing differentiation of immune cells comprises administering a CHP as disclosed herein or a composition as disclosed herein to a subject. In an embodiment, the step of administering is performed by injection. In an embodiment, the step of administering is performed by micro-dermal injection. In an embodiment, the step of administering is performed by topical application.

In a further aspect, a method of downregulating immune cells comprises administering a CHP as disclosed herein or a composition as disclosed herein to a subject. The immune cells may be one or more of T-Cells, B-Cells, Natural Killer Cells, Macrophages, Monocytes, and Neutrophils. In an embodiment, the step of administering is performed by injection. In an embodiment, the step of administering is performed by micro-dermal injection. In an embodiment, the step of administering is performed by topical application.

In another aspect, a method of preventing the differentiation of immune cells comprises administering a CHP as disclosed herein or a composition as disclosed herein to a subject. The immune cells may be one or more of T-Cells, B-Cells, Natural Killer Cells, Macrophages, Monocytes, and Neutrophils. In an embodiment, the step of administering is performed by injection. In an embodiment, the step of administering is performed by micro-dermal injection. In an embodiment, the step of administering is performed by topical application.

In an aspect, a method of preventing or treating dry eye disease in a subject comprises administering a CHP as disclosed herein or a composition as disclosed herein to a subject. In an embodiment, the administering is performed by local injection, intravenous injection, topical application, or physical application. In a further embodiment, the administering is performed by local injection. In a further embodiment, the administering is performed by intravenous injection. In a further embodiment, the administering is performed by topical application. In a further embodiment, the administering is performed by physical application.

In another embodiment of the method of preventing or treating dry eye disease in a subject, the CHP or composition is administered as a putty, mesh, patch, adhesive, cream, sutures, gel or eyedrops. In a further embodiment, the CHP or composition is administered as a putty. In a further embodiment, the CHP or composition is administered as a mesh. In a further embodiment, the CHP or composition is administered as a patch. In a further embodiment, the CHP or composition is administered as an adhesive. In a further embodiment, the CHP or composition is administered as a cream. In a further embodiment, the CHP or composition is administered as one or more sutures. In a further embodiment, the CHP or composition is administered as a gel. In a further embodiment, the CHP or composition is administered as eyedrops.

In an aspect, a method of preventing or treating atopic dermatitis in a subject comprises administering a CHP as disclosed herein or a composition as disclosed herein to a subject. In an embodiment, the administering is performed by local injection, intravenous injection, topical application, or physical application. In a further embodiment, the administering is performed by local injection. In a further embodiment, the administering is performed by intravenous injection. In a further embodiment, the administering is performed by topical application. In a further embodiment, the administering is performed by physical application.

In another embodiment of the method of preventing or treating atopic dermatitis in a subject, the CHP or composition is administered as a putty, mesh, patch, adhesive, cream, sutures, gel or eyedrops. In a further embodiment, the CHP or composition is administered as a putty. In a further embodiment, the CHP or composition is administered as a mesh. In a further embodiment, the CHP or composition is administered as a patch. In a further embodiment, the CHP or composition is administered as an adhesive. In a further embodiment, the CHP or composition is administered as a cream. In a further embodiment, the CHP or composition is administered as one or more sutures. In a further embodiment, the CHP or composition is administered as a gel.

In an aspect, a method of preventing or treating osteoarthritis in a subject comprises administering a CHP as disclosed herein or a composition as disclosed herein to a subject. In an embodiment, the administering is performed by local injection, intravenous injection, topical application, or physical application. In a further embodiment, the administering is performed by local injection. In a further embodiment, the administering is performed by intravenous injection. In a further embodiment, the administering is performed by topical application. In a further embodiment, the administering is performed by physical application.

In another embodiment of the method of preventing or treating osteoarthritis in a subject, the CHP or composition is administered as a putty, mesh, patch, adhesive, cream, sutures, gel or eyedrops. In a further embodiment, the CHP or composition is administered as a putty. In a further embodiment, the CHP or composition is administered as a mesh. In a further embodiment, the CHP or composition is administered as a patch. In a further embodiment, the CHP or composition is administered as an adhesive. In a further embodiment, the CHP or composition is administered as a cream. In a further embodiment, the CHP or composition is administered as one or more sutures. In a further embodiment, the CHP or composition is administered as a gel.

In an aspect, a method of preventing or treating rheumatoid arthritis in a subject comprises administering a CHP as disclosed herein or a composition as disclosed herein to a subject. In an embodiment, the administering is performed by local injection, intravenous injection, topical application, or physical application. In a further embodiment, the administering is performed by local injection. In a further embodiment, the administering is performed by intravenous injection. In a further embodiment, the administering is performed by topical application. In a further embodiment, the administering is performed by physical application.

In another embodiment of the method of preventing or treating rheumatoid arthritis in a subject, the CHP or composition is administered as a putty, mesh, patch, adhesive, cream, sutures, gel or eyedrops. In a further embodiment, the CHP or composition is administered as a putty. In a further embodiment, the CHP or composition is administered as a mesh. In a further embodiment, the CHP or composition is administered as a patch. In a further embodiment, the CHP or composition is administered as an adhesive. In a further embodiment, the CHP or composition is administered as a cream. In a further embodiment, the CHP or composition is administered as one or more sutures. In a further embodiment, the CHP or composition is administered as a gel.

In an aspect, a method of preventing or treating diabetic lesions in a subject comprises administering a CHP as disclosed herein or a composition as disclosed herein to a subject. In an embodiment, the administering is performed by local injection, intravenous injection, topical application, or physical application. In a further embodiment, the administering is performed by local injection. In a further embodiment, the administering is performed by intravenous injection. In a further embodiment, the administering is performed by topical application. In a further embodiment, the administering is performed by physical application.

In another embodiment of the method of preventing or treating diabetic lesions in a subject, the CHP or composition is administered as a putty, mesh, patch, adhesive, cream, sutures, gel or eyedrops. In a further embodiment, the CHP or composition is administered as a putty. In a further embodiment, the CHP or composition is administered as a mesh. In a further embodiment, the CHP or composition is administered as a patch. In a further embodiment, the CHP or composition is administered as an adhesive. In a further embodiment, the CHP or composition is administered as a cream. In a further embodiment, the CHP or composition is administered as one or more sutures. In a further embodiment, the CHP or composition is administered as a gel.

In an aspect, a method of preventing or treating psoriasis in a subject comprises administering a CHP as disclosed herein or a composition as disclosed herein to a subject. In an embodiment, the administering is performed by local injection, intravenous injection, topical application, or physical application. In a further embodiment, the administering is performed by local injection. In a further embodiment, the administering is performed by intravenous injection. In a further embodiment, the administering is performed by topical application. In a further embodiment, the administering is performed by physical application.

In another embodiment of the method of preventing or treating psoriasis in a subject, the CHP or composition is administered as a putty, mesh, patch, adhesive, cream, sutures, gel or eyedrops. In a further embodiment, the CHP or composition is administered as a putty. In a further embodiment, the CHP or composition is administered as a mesh. In a further embodiment, the CHP or composition is administered as a patch. In a further embodiment, the CHP or composition is administered as an adhesive. In a further embodiment, the CHP or composition is administered as a cream. In a further embodiment, the CHP or composition is administered as one or more sutures. In a further embodiment, the CHP or composition is administered as a gel.

In an aspect, a method of increasing immune checkpoints in a subject comprises administering a CHP as disclosed herein or a composition as disclosed herein to a subject. In an embodiment, the administering is performed by local injection, intravenous injection, topical application, or physical application. In a further embodiment, the administering is performed by local injection. In a further embodiment, the administering is performed by intravenous injection. In a further embodiment, the administering is performed by topical application. In a further embodiment, the administering is performed by physical application.

In another embodiment of the method of increasing immune checkpoints in a subject, the CHP or composition is administered as a putty, mesh, patch, adhesive, cream, sutures, gel or eyedrops. In a further embodiment, the CHP or composition is administered as a putty. In a further embodiment, the CHP or composition is administered as a mesh. In a further embodiment, the CHP or composition is administered as a patch. In a further embodiment, the CHP or composition is administered as an adhesive. In a further embodiment, the CHP or composition is administered as a cream. In a further embodiment, the CHP or composition is administered as one or more sutures. In a further embodiment, the CHP or composition is administered as a gel. In a further embodiment, the CHP or composition is administered as eyedrops.

Each description and embodiment disclosed in the present disclosure may also be applied to other descriptions and embodiments. That is, all combinations of various elements disclosed in the present disclosure fall within the scope of the present application. Further, the scope of the present application is not limited by the specific description below. In addition, those skilled in the art will recognize or be able to ascertain using no more than routine experimentation, many equivalents to the specific aspects of the present application described herein. Further, these equivalents should be interpreted to fall within the scope of the present application.

### EXAMPLES

With reference to the appended drawings, exemplary embodiments of the present disclosure will be described in detail below. To aid in understanding the present disclosure, like numbers refer to like elements throughout the description of the figures, and the description of the same elements will be not reiterated.

Synthesis and In Vitro Characterization of Anti-Inflammatory Bioactive Collagen hybridizing peptides (CHPs)

All peptide sequences, including CHPs, are synthesized on a TentaGel R RAM resin (Peptides International, RTS-9995-PI) with standard Fmoc-chemistry on a CS-136X auto-synthesizer (CSBio) using Fmoc-Gly-OH, Fmoc-Pro-OH, Fmoc-Hyp(tBu)-OH (EMD Millipore), and Fmoc-cis-4-fluoro-Pro-OH (Chem-Impex, 14494) as the main amino acid residues. Other residues include Fmoc-Lys(boc)-OH, Fmoc-Phe-OH, Fmoc-Ala-OH, Fmoc-Arg(Pbf)-OH, Fmoc-Leu-OH, Fmoc-Gln(Trt)-OH, Fmoc-Asp-OH, and Fmoc-Glu(OtBu)-OH. In addition, any other commercially available fmoc-, boc-, or Z-protected amino acids may be used in the bioactive sequences, non-natural amino acids may also be incorporated to enhance desirable traits of the overall peptide, such as increased binding (aza-glycine), hydrophilicity, immune evasion, stability, etc. HATU (Aapptec) and 1-hydroxy-7-azabenzo-triazole (HOAt, Aapptec) are used as the coupling reagents. Piperidine (20% by volume) in N-methyl-2-pyrrolidone is used to remove the Fmoc protective group. Five molar equivalents of amino acid residues (0.119 M) and coupling reagents (0.114 M) in dimethylformamide are used in each coupling reaction. Resins are treated with trifluoroacetic acid (TFA)/triisopropylsilane (TIS)/water (95:2.5:2.5) for 2 h, and the crude peptides are precipitated by adding excess cold ether to the TFA solution. The peptides are purified by reverse phase high-performance liquid chromatography (RP-HPLC) on an Agilent Zorbax SB C-18 column using a linear gradient mixture of water (0.1% TFA) and acetonitrile (0.1% TFA) (5-35% acetonitrile in 30 min) as the mobile phase. The molecular weights of the purified peptides are verified using electrospray ionization time of flight (ESI-TOF) MS. Peptide purity is measured by HPLC.

To ensure all synthesized CHPs retain their ability to bind with denatured collagen, they are evaluated for their triple-helicity using a CD spectrometer to identify their melting temperatures (Tm) for dissociation. A gelatin binding assay and tissue histology are used to confirm the CHPs specifically bind to denatured collagen.

### In Vitro Assessment of Effect of LAIR-1 Bioactive CHPs on T-Cell Activation and Inhibition

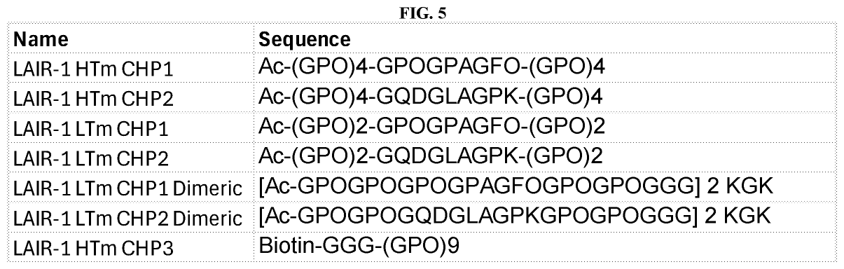

To evaluate the effects of the LAIR-1 anti-inflammatory bioactive CHPs, exemplary CHPs are tested (see FIG. 5 for a listing of the CHPs used in in vitro testing).

### Plate Assay for LAIR-1 Binding

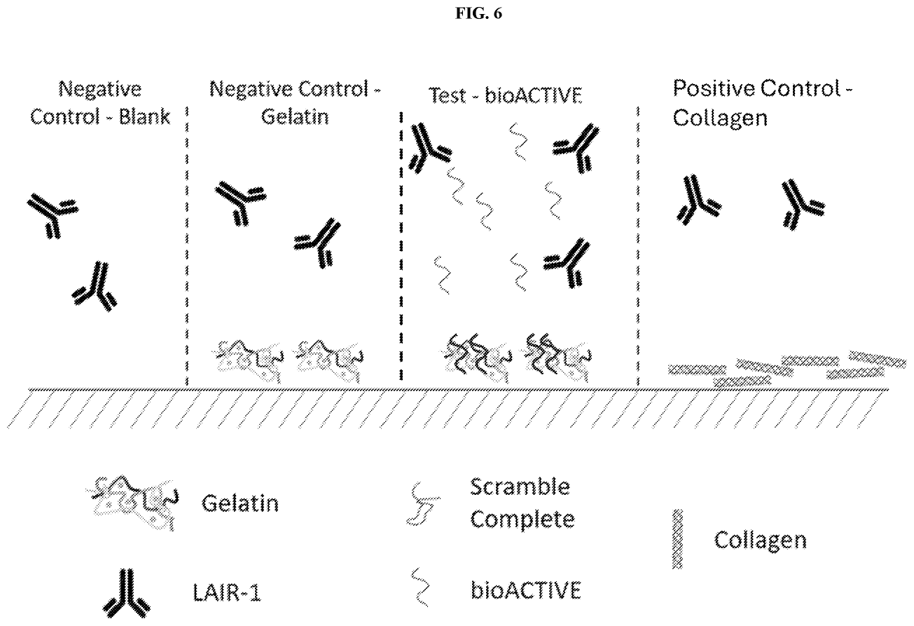

First, a LAIR-1 plate binding assay to detect LAIR-1 binding to LAIR-1 bioactive CHPs (both monomeric and dimeric) was carried out (see FIG. 6 for a depiction of the experimental setup). This was a plate assay developed to allow the bioactive CHPs to bind to gelatin for detection of LAIR1 binding by using a second antibody to detect bound LAIR-1 protein.

A 96-well black opaque Nunc Amino flat-bottom plate was coated with 100 g/mL NEUTRAL pH (pH 7) protein solution (gelatin from porcine skin or intact collagen from human placenta) in PBS per well. Negative control (blank) wells received neither the gelatin nor collagen protein solutions to yield open surface. Negative control (gelatin) wells and bioactive CHP test wells both received gelatin. Positive control wells received the intact human collagen. The plate was then covered with packing tape to reduce evaporation and spillover and incubated on a shaker at room temperature for 2 hours.

After incubation, each well of the plate was washed with successive washes of 200 μL PBS for a total of 4 washes, for approximately 5 minutes. The last wash was discarded.

Wells were then fixed with 150 μL of 100% ice-cold methanol (stored in −20 C) and incubated at −20° C. for 30 minutes. After incubation, each well of the plate was washed with successive washes of 200 μL PBS for a total of 4 washes, for approximately 5 minutes. The last wash was discarded.

The plate was then incubated with IM TRIS-HCL pH 7.4 for 30 minutes at room temperature on shaker (200 μL per well); this was done with the intention of blocking any remaining reactive groups on the surface of the amino capture plates. Again, each well of the plate was washed with successive washes of 200 μL PBS for a total of 4 washes, for approximately 5 minutes. The last wash was discarded.

Each well of the plate was then blocked with 200 μL 1% BSA in 1×PBS pH 7.4 for 30 minutes at room temperature. After blocking, each well of the plate was washed with successive washes of 200 μL PBS for a total of 4 washes, for approximately 5 minutes. The last wash was discarded.

The bioACTIVE CHP peptide solutions were dissolved in 1×PBS at a concentration of 500M and heated at 80° C. for 10 minutes to release CHPs into the monomeric form required to bind to denatured collagen. The peptide solutions were then quenched in ice water until cool to touch (room temperature). Then, 50 μL of CHP in 1×PBS solution were added to each test well of the plate (negative control (blank) wells, negative control (gelatin) wells, and positive control (collagen) wells did not receive CHPs).

The plate was covered with tape and incubated overnight to allow CHPs to bind at specified temperature. The final CHP solution was removed, and each well was washed with successive washes of 200 μL cold PBS for a total of 3 washes, for 10 minutes. The last wash was discarded.

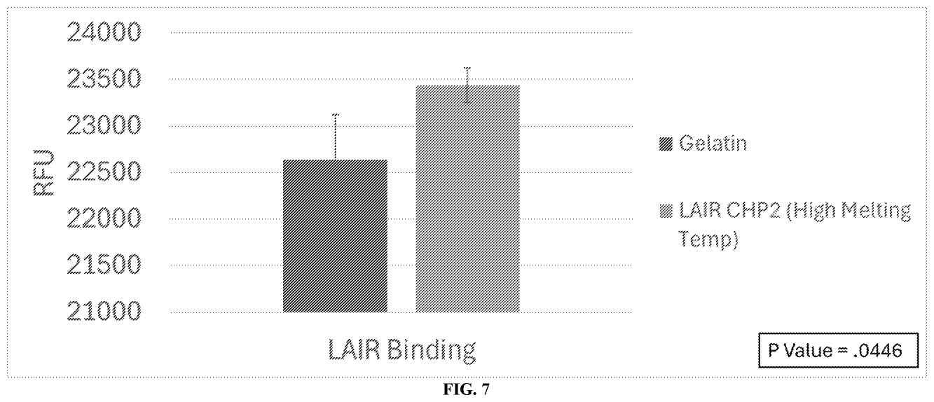

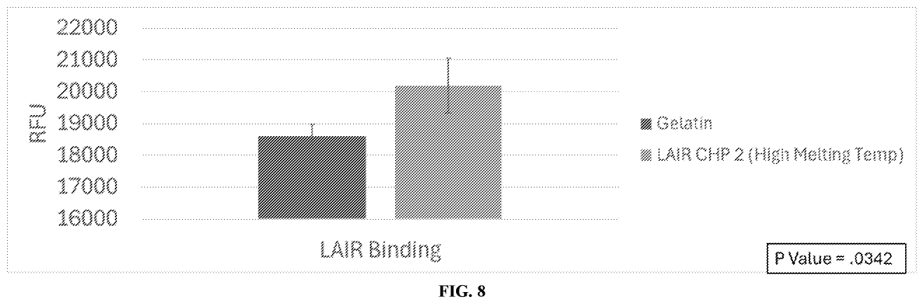

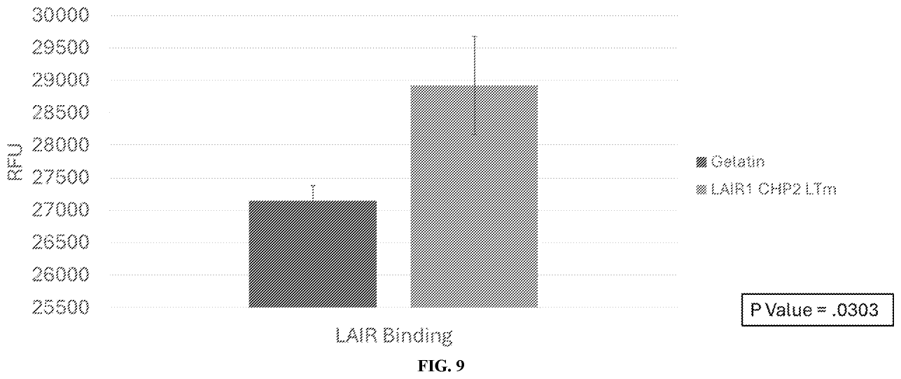

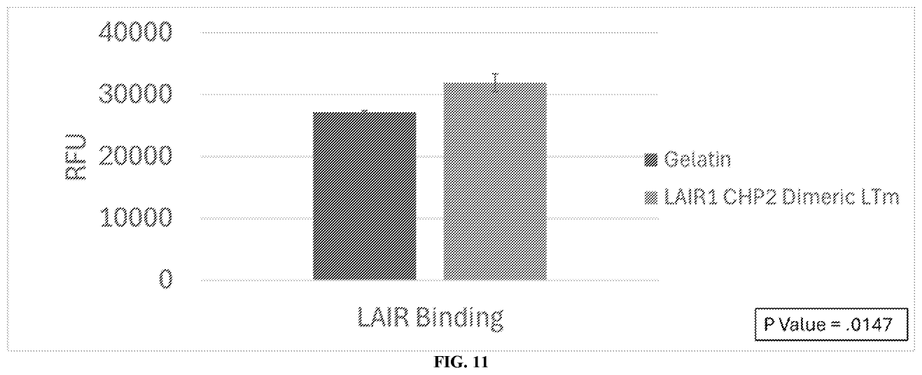

Recombinant human LAIR-1 Fc Chimera protein in 1% BSA in PBS (5 μg/mL final) was then added to each well (100 μL per well). The plate was incubated for one hour at room temperature (FIG. 7) or at 37° C. (FIG. 8), or for two hours at 4° C. (FIGS. 9 and 11), to allow LAIR-1 protein to bind.

The LAIR solution was then removed from the wells, which were then washed with successive washes of 200 μL cold PBS for a total of 4 washes, for 10 minutes. The last wash was discarded.

Anti-human IgG Alexa Fluor 488 antibody in 1% BSA in PBS (5 μg/mL final) was added to each well (100 μL per well). The plate was then incubated for one hour at room temperature or at 37° C., or for two hours at 4° C. covered in foil. The antibody solution was removed from the wells, which were then washed with successive washes of 200 μL cold PBS for a total of 4 washes, for 10 minutes. The last wash was discarded.

All liquid was then removed from each of the wells to allow them to dry. Once dried, each well was imaged on a Spectra Max iD3 plate reader for fluorescent signal with an excitation of 480 nm and detection at 520 nm. A total of 32 evenly spaced reads were captured and averaged per well.

FIG. 7 depicts the results of a plate binding assay at room temperature incubation for a LAIR-1 monomeric bioactive CHP referenced as LAIR-1 HTm CHP2 (SEQ ID NO: 2, wherein x=4 and y=4) having a high melting temperature. As shown in the graph, LAIR-1 HTm CHP2 monomer increases LAIR-1 binding by 3.5% as indicated by Relative Fluorescence Units (RFUs) with room temperature incubation over negative (gelatin) controls (P-value=0.0446 from a one-tailed, type-2 T-test). Three replicates per group were used.

FIG. 8 depicts the results of a plate binding assay at 37° C. incubation (approximately human body temperature) for a LAIR-1 monomeric bioactive CHP referenced as LAIR-1 HTm CHP2 (SEQ ID NO: 2, wherein x=4 and y=4) having a high melting temperature. As shown in the graph, LAIR-1 HTm CHP2 monomer increases LAIR-1 binding by 8.5% as indicated by RFUs with 37° C. incubation over negative (gelatin) controls (P-value=0.0342 from a one-tailed, type-2 T-test). Three replicates per group were used. Without being bound by theory, it is believed that the difference in binding demonstrated by LAIR-1 HTm CHP2 between the room temperature incubation assay and the 37° C. incubation assay is caused by an increase in the amount of available favorable attachment sites in the gelatin test wells and reduced CHP self-trimerization at 37° C. (approximate human body temperature).

FIG. 9 depicts the results of a plate binding assay at 4° C. incubation for a LAIR-1 monomeric bioactive CHP referenced as LAIR-1 LTm CHP2 (SEQ ID NO: 2, wherein x=2 and y=2) having a low melting temperature. As shown in the graph, LAIR-1 LTm CHP2 monomer increases LAIR-1 binding by 6.5% as indicated by RFUs with 4° C. incubation over negative (gelatin) controls (P-value=0.0303 from a one-tailed, type-2 T-test). Three replicates per group were used. The incubation temperature was selected due to the low melting temperature of LAIR-1 LTm CHP2.

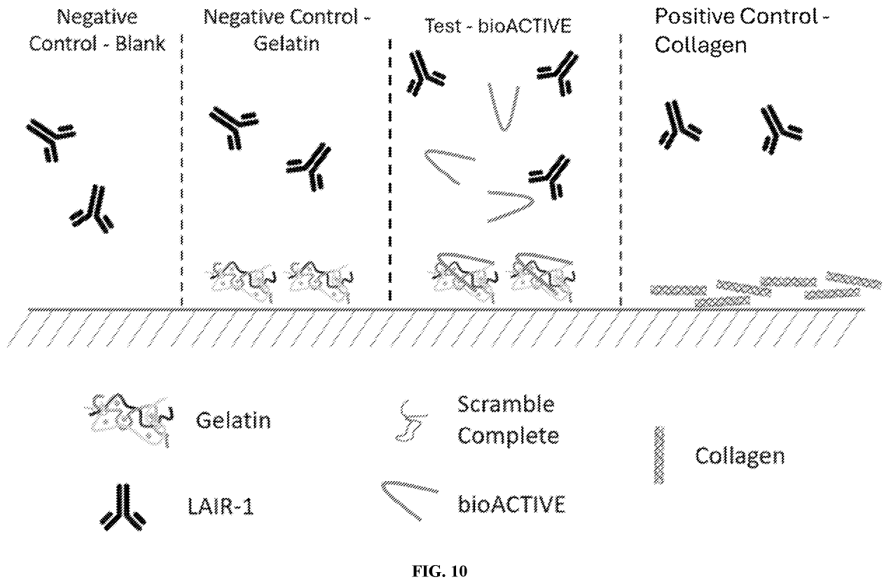

FIG. 10 depicts the experimental design of a LAIR-1 plate binding assay to detect LAIR-1 binding to LAIR-1 dimeric bioactive CHPs.

FIG. 11 depicts the results of a plate binding assay at 4° C. incubation for a LAIR-1 dimeric bioactive CHP referenced as LAIR-1 LTm CHP2 Dimeric (two copies of SEQ ID NO: 2, wherein x=2 and y=2, each copy attached to SEQ ID NO:105 as linker, wherein z=1) with a low melting temperature in accordance with embodiments disclosed herein. As shown in the graph, LAIR-1 LTm CHP2 Dimeric increases LAIR-1 binding by 17.6% as indicated by RFUs with 4° C. incubation over negative (gelatin) controls (with a P-value of 0.0147 from a one-tailed, type-2 T-test). Three replicates per group were used. The incubation temperature was selected due to the low melting temperature of LAIR-1 LTm CHP2 Dimeric.

### In Vitro Immune Cell Activation Assay

To evaluate the effects that the anti-inflammatory bioactive CHPs (e.g., CHPs including LAIR-1 sequence) have, several in vitro parameters are assessed. First, immune cells (e.g., Jurkat T-cells, CD4+ cells, CD8+ cells, naïve T-cell lines, macrophages, etc.) are cultured in suspension using the appropriate cell media and additives. Once cells have proliferated, they are placed on tissue culture well-plates with or without gelatin (denatured collagen). The thickness of crosslinked gelatin ranges based on the volume of gelatin solution added which is between 50-250 μl per well. The gelatin supplies binding sites for CHP binding; thus, the well-plates with gelation serve as the experimental group, and the blank wells (which include PBS+ Media or Media only) serve as controls.

Next, anti-inflammatory bioactive CHPs are added to the experimental and control wells, and are replenished daily when the cell media is exchanged. ELISA is performed on the supernatant taken from each well (both experimental and control) and assessed for IL-1, IL-6, IL-7, IL-8, TNF-α, IFN-γ, IL-17, IL-22, and IL-27. The wells treated with gelatin/collagen and CHPs demonstrate a decrease in the levels of these inflammatory cytokines compared to control wells.

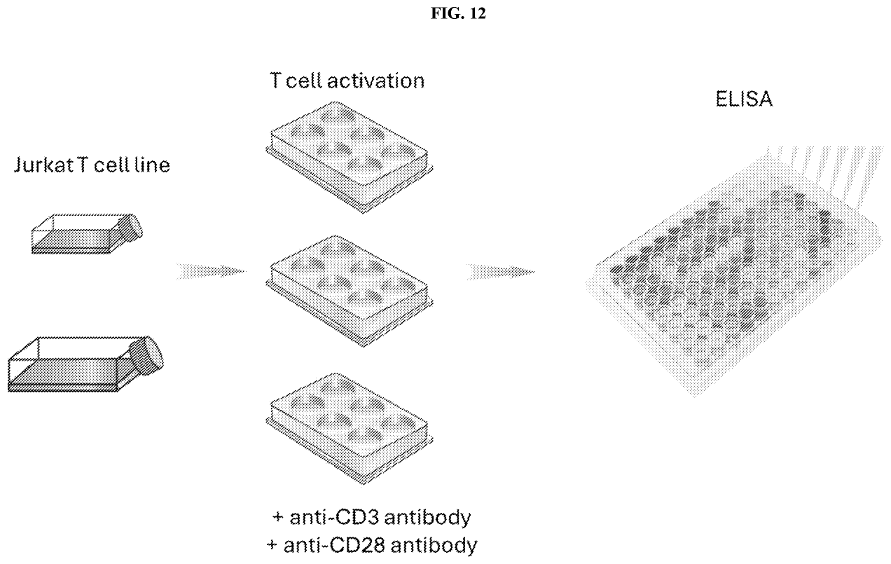

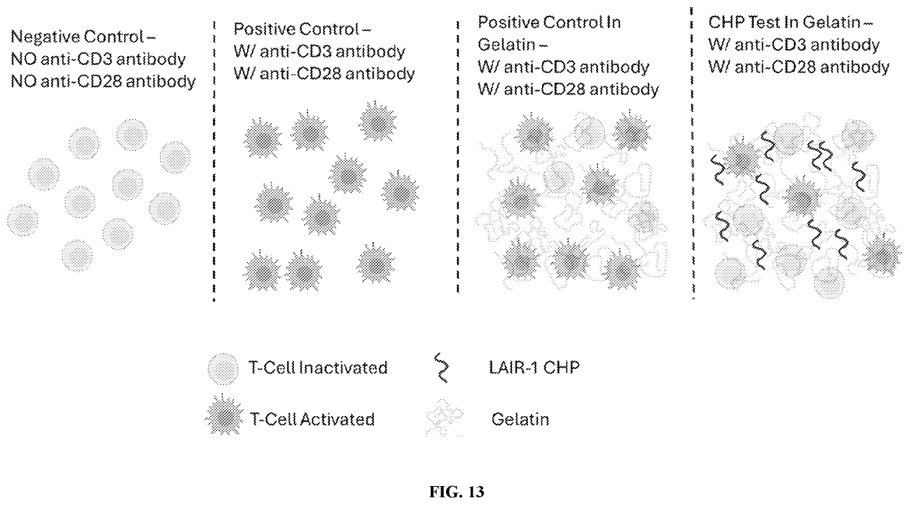

A particular in vitro activation assay was carried out to evaluate the effects that the anti-inflammatory bioactive LAIR-1 CHPs have on T-cells. A schematic of this T-cell activation/inhibition assay is shown in FIG. 12. Details regarding the experimental conditions for the T-cell assay with Jukat T-cells are depicted in FIG. 13. As shown, Jukat T-cells were incubated with activating antibodies against CD3 and CD28 receptors to initiate activation. The negative control set of T-cells did not receive anti-CD3 and anti-CD28 antibodies. T-cell activation was then assessed by ELISA for IL-2 secretion in media supernatant. Activated T-cells express high levels of IL-2.

Secreted IL-2 was measured for the following conditions: negative control which were unactivated T cells; positive control T cells which received anti-CD3 and anti-CD28 antibodies; positive control t cells which received stimulating antibodies in the presence of soluble gelatin; and the CHP test condition which received stimulating antibodies in the presence of LAIR1-CHPs.

For the T-cell activation/inhibition experimental protocol, three monomeric CHPs (LAIR-1 HTm CHP1 [SEQ ID NO: 87, wherein x=4 and y=4], LAIR-1 HTm CHP2 [SEQ ID NO: 2, wherein x=4 and y=4], and LAIR-1 HTm CHP3 [SEQ ID NO: 106]) were tested for T-cell activation/inhibition. First, CHPs were incubated with the gelatin under test conditions for at least 24 hours at 37° C. The final gelatin concentration in the media for incubation was 0.5 mg/mL.

Next, the activation wells (positive controls and CHP test conditions) of a Nunc Cell Culture Treated 6 well plate were coated with the anti-CD3 antibody (Anti-Human CD3 (OKT3)) at 3 μg/mL in 2 mL. The negative control well was not coated.

The plate was then incubated at 37° C. in 5% CO₂ for 2 hours. The anti-CD3 mixture was then aspirated (not rinsed) from each well to which it was coated.

900,000 Jurkat T-Cells (E6-1 from ATCC) were added to each plate in media with their respective treatments and CD28 antibody (Anti-Human CD28 Monoclonal antibody (CD28.2)) at 3 μg/mL in a total volume of 3 mL. The complete media contained RPMI 1640 medium, fetal bovine serum, L-glutamine, and penicillin-streptomycin.

The resulting 6-well plate included:
Anti-CD28 (Anti-Human CD28 Monoclonal antibody) at 3 μg/mL for all wells except for negative control.
CyA (Cyclosporine) at 5 μM for known treatment control in the presence of both Anti-CD28 and Anti-CD3 in one well.
Gelatin at 0.5 mg/mL for wells containing T-Cells in presence of gelatin.
Gelatin at 0.5 mg/mL with CHPs at a final concentration of 500 μM in LAIR-1 HTm CHP1 and LAIR-1 HTm CHP2 test wells, and 100 μM in LAIR-1 HTm CHP3 test wells.

The 6-well plate with T-cells was then incubated for 4 days at 37° C. for activation of the T-cells. At day 3, 500 μl of cell media from each well was removed for testing with the IL-2 ELISA kit. The 6-well plate was incubated for an additional day for further T-cell activation at 37° C., for a total of 4 days. Then, 500 μl of cell media from each well was removed to complete the IL-2 ELISA kit, which was run to the kit manufacturer's specifications.

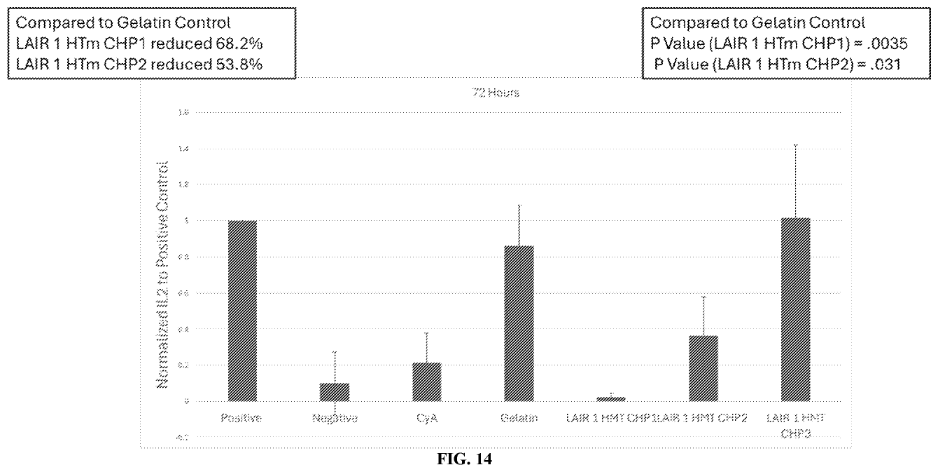

FIG. 14 depicts the results of an in vitro T-cell activation assay at 37° C. via detection of IL-2 levels by ELISA at 72 hours post-stimulation. Gelatin (damaged collagen) acts as a second positive control, as CHPs require damaged collagen to localize and bind. The well with Cyclosporin A (CyA) inhibited T-cell activation by halting production of interleukins, including IL2. When compared to the gelatin positive control LAIR-1 HTm CHP1 reduced IL2 production by 68.2% (P-value=0035) and LAIR 1 HTm CHP2 reduced IL 2 production by 53.8% (P-value=0.031). LAIR 1 HTm CHP 3 did not have an effect on the IL 2 production.

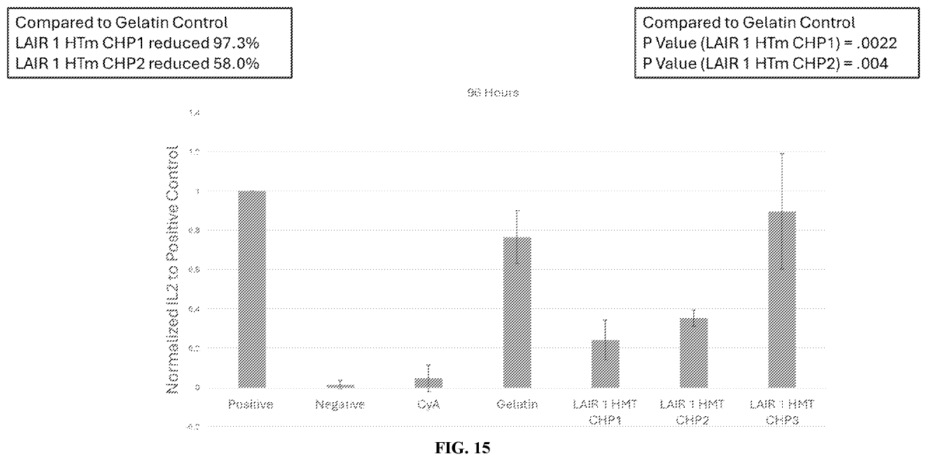

FIG. 15 depicts the results of an in vitro T-cell activation assay at 37° C. via detection of IL-2 levels by ELISA at 96 hours post-stimulation. T-cell activation was confirmed by high levels of IL-2 at 96 hours post-stimulation. Gelatin (damaged collagen) acts as a second positive control, as CHPs require damaged collagen to localize and bind. The well with Cyclosporin A (CyA) inhibited T-cell activation by halting production of interleukins, including IL2. When compared to the gelatin positive control LAIR 1 HTm CHP1 reduced IL 2 production by 97.3% (P Value=0.0022) and LAIR-1 HTm CHP2 reduced IL 2 production by 58.0% (P Value=. 004). LAIR-1 HTm CHP3 did not have an effect on the IL 2 production.

### Ex Vivo Characterizations in Atopic Dermatitis, Psoriasis, and Dry-Eye Disease Models

Ex vivo characterizations are also carried out to confirm the results of in vitro characterization of the anti-inflammatory bioactive CHPs in different immune cell lines cultured in suspension (e.g., Jurkat T-cells, macrophages).

### Psoriasis/Atopic Dermatitis Models

The “Inflammaskin” model for psoriasis from GenoSkin is used as the ex vivo inflammation model for atopic dermatitis. Since the mechanisms of action between atopic dermatitis/eczema and psoriasis are very similar, and these conditions have almost identical symptoms of red, itchy, flaky, and inflamed skin lesions, such a model provides an suitable method to test the anti-inflammatory bioactive CHPs and their modulation of the inflammatory pathway. This model is used to evaluate both the therapeutic response and the prophylactic effects of the CHPs.

For therapeutic evaluation, the Inflammaskin model is allowed to progress for 3-4 days without treatment before topical application of CHPs for another 3-4 days. For the prophylactic model, CHPs are administered by topical application on top of the skin model as the model is being challenged with the activation cocktail. The HypoSkin model without T-cell activation is used as a healthy control.

Evaluation of the skin models is based on histopathological analysis at the end of the study (Day 7) and quantitative analysis of skin structure integrity is performed through attribution of a score depending on defects detected in the models on H&E stained sections:
0: Uncultured skin without any changes
1: Early signs of keratinocytes separation (spongiosis)
2: Advanced separation and volume increase of keratinocytes (hypertrophy)/Early signs of dermis disorganization
3: Nuclei condensation (pyknosis)/Advanced dermis disorganization
4: Cytoplasms vacuolation
5: Stratum basale detachment from dermis/Release of stratum spinosum

Additionally, the supernatant is collected and run on ELISA to evaluate levels of inflammatory cytokines similar to the protocol for the in vitro cell culture model.

### Dry-Eye Disease Models

For dry-eye disease (DED) the ex vivo eye irritation test (EVEIT) is employed. This model measures a substance's ability to cause corneal injury and assesses reversibility. Rabbit corneas are extracted from rabbit eyes, and the cells are sustained for 72 hours. Injury to the epithelial barrier function is detected using a sodium fluorescein stain.

The therapeutic effect of the anti-inflammatory CHPs is probed using a comparison between corneas treated with the bioactive CHPs and corneas dosed with PBS (PBS sham models). The bioactive CHPs or the PBS sham are applied to the corneas 4-6 times a day. Then the cornea barrier integrity is evaluated using optical coherence tomography (OCT) at multiple times during the experiment to noninvasively assess epithelium and stromal injury and recovery. At the end of the experiment, corneas are fixed and sectioned for histopathological examination of the cornea repair using H&E and fluorescently labeled CHPs for detecting remodeling collagen. These sections are evaluated by a pathologists for accuracy and consistency. In addition, the cells are collected from one corneal section per group to undergo rtPCR for the evaluation of inflammatory markers. The diseased models are compared to the PBS sham models.

### In Vivo Characterizations in Atopic Dermatitis Models and Dry-Eye Disease Models

### In Vivo Dry-Eye Disease Models

Since there are no perfect models that effectively mimic all conditions of dry-eye disease (DED), several in vivo models can be utilized to ensure a thorough understanding of the anti-inflammatory effects of the bioactive CHPs as a therapeutic treatment for DED. An airflow and scopolamine model is used. This model mimics the desiccating environmental stress on the ocular surface of the eyes. This is often considered one of the initiating factors for DED. This model exposes C57BL/6 female mice to evaporative conditions via constant low-humidity air flow aimed at the face. The longer the exposure, the more prolonged the decreased tear secretion there is. In addition to drying the ocular surface from environmental factors, this model also inhibits the muscarinic receptors using subcutaneous administration of scopolamine. This reduces the tear secretion and together this model represents a good mimic for aqueous-deficient DED featuring reduction in tear production, tear film stability, and increase in apoptosis of ocular surface epithelium and cytokine levels.

Two studies using this mouse model are carried out to test the therapeutic effect versus the prophylactic ability of anti-inflammatory bioactive CHPs. Three groups of mice are used—a group for each bioactive CHP tested, a group for a sham (PBS) control, and a group for healthy control. For the therapeutic evaluation study, the experimental groups of mice are induced to develop symptoms of DED by placing them in the controlled environmental chamber (CEC) with a relative humidity below 15%, airflow of 10 L/min, and a constant temperature of 21° C. to 23° C. for 14 days, while subcutaneously administering 0.1 mL of 5 mg/mL scopolamine hydrobromide three times per day (9 AM, 1 PM, and 5 PM) on the dorsal surface of the mice. Once DED symptoms are established, 0.1 ml of 5 nmol doses bioactive CHPs were delivered by intra-orbital injection every day; however, other methods that can be tested include an eye-drop delivery and systemic delivery via tail-vein injections.

The therapeutic response of CHPs is then measured by how quickly the mice returned to normal once placed back into normal vivarium conditions and halting scopolamine injections as measured with corneal fluorescein staining (CFS), which is used to evaluate corneal epithelial damage caused by DED. A dose of 1 μL of 2.5% fluorescein is applied into the lateral conjunctival sac of the mice, and 3 minutes later their corneas are examined using a slit-lamp biomicroscope (Topcon SL-D7; Topcon Corp., Tokyo, Japan) under cobalt blue light. Punctate staining is recorded in a masked fashion using the National Eye Institute grading system, scoring 0 to 3 for each of five areas of the cornea: central, superior, inferior, nasal, and temporal.

In addition, the tears from each mouse are collected and run on ELISA to evaluate the inflammatory response by measuring levels of IL-1, IL-6, IL-7, IL-8, TNF-α, IFN-γ, IL-17, IL-22, and IL-27.

For the prophylactic study, CHPs are administered daily as the mice underwent the same procedure as above. The same endpoints and inflammatory evaluations are also implemented.

### In Vivo Atopic Dermatitis Models

For an in vivo atopic dermatitis (eczema) model, the oxazolone induced model in SKH-1 hairless mice is used. With this model the disease response can be monitored via ear measurements (in-life), histopathological analysis, and cytokine analysis with ELISA. Anti-inflammatory bioactive CHPs are tested in comparison to a control peptide using two routes of CHP administration. Four groups of mice are used—a group for each of the two administration methods for delivery of the bioactive CHPs, a group for a PBS control, and a group for healthy control in mice that do not have atopic dermatitis.

One administration method for delivering anti-inflammatory bioactive CHPs is a systemic tail-vein injection as a 1-5 nmol single bolus every three days. The other method evaluated is topical application of CHPs at the site of the eczema lesions. These different methods of administration are evaluated for the therapeutic effects the CHPs have on the inflammatory response, and the administration methods are compared to determined which route of administration is more effective. Evaluation is performed by measuring the sizes of eczema lesions on the ears of the nude mice. During treatment with the CHPs, the size of the lesions decrease.

At the end of the experiment, the mice are sacrificed and histology is performed on the skin. Unaffected areas are compared to the sites of lesions from the same mouse, but comparisons are also made across each experimental group. Skin is stained with H&E, Masson's Trichrome, and a biotin-labeled CHP. The sections are evaluated by veterinary pathologists to assess the degree of inflammation and the beneficial effects from the anti-inflammatory bioactive CHPs. Blood is also collected and run on ELISA to test for inflammatory biomarkers IL-1, IL-6, IL-7, IL-8, TNF-α, IFN-γ, IL-17, IL-22, and IL-27.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A collagen hybridizing peptide (CHP), wherein the CHP has a sequence represented by Formula I: 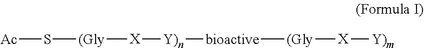
in which Ac is an acetyl capping group; S is zero or more spacer molecules; X is proline or modified proline; Gly is glycine; Y is hydroxyproline or any other amino acid; n is a number from 1 to 20; m is a number from 1-20; and
the bioactive is an amino acid sequence from 3 to 27 amino acid residues having bioactivity, and comprises at least one sequence selected from the group consisting of a leukocyte-associated immunoglobulin-like receptor sequence (LAIR-1 and/or LAIR-2), a G6b-B recognition site sequence, an OSCAR domain sequence, a GPVI sequence, and a FcR-γ domain sequence.

2. The CHP according to claim 1, wherein the at least one bioactive comprises a LAIR-1 receptor sequence.

3. The CHP according to claim 1, wherein the at least one bioactive comprises a LAIR-2 receptor sequence.

4. The CHP according to any one of the preceding claims, wherein the at least one bioactive comprises a G6b-B recognition site sequence.

5. The CHP according to any one of the preceding claims, wherein the at least one bioactive comprises a FcR-γ domain sequence.

6. The CHP according to any one of the preceding claims, wherein the at least one bioactive comprises an OSCAR domain sequence.

7. The CHP according to any one of the preceding claims, wherein the at least one bioactive comprises a GPVI sequence.

8. The CHP according to any one of the preceding claims, wherein the at least one bioactive comprises a LAIR-1 and/or LAIR-2 receptor sequence comprising a sequence having at least 85% sequence identity to any one of SEQ ID NO: 1-27, 56-64, 93, 94, and 98-100.

9. The CHP according to any one of the preceding claims, wherein the at least one bioactive comprises a G6b-B recognition site sequence comprising a sequence having at least 85% sequence identity to any one of SEQ ID NO: 28-46, 95, and 96.

10. The CHP according to any one of the preceding claims, wherein the at least one bioactive comprises a FcR-γ domain sequence comprising a sequence having at least 85% sequence identity to any one of SEQ ID NO: 47-55 and 97.

11. The CHP according to any one of the preceding claims, wherein the at least one bioactive comprises a GPVI domain sequence comprising a sequence having at least 85% sequence identity to any one of SEQ ID NO: 65-83 and 101-103.

12. The CHP according to any one of the preceding claims, wherein the at least one bioactive comprises an OSCAR domain sequence comprising a sequence having at least 85% sequence identity to any one of SEQ ID NO: 84-92 and 104.

13. The CHP according to any one of the preceding claims, wherein the CHP comprises a sequence selected from the group consisting of SEQ ID NO: 1-104.

14. The CHP according to any one of the preceding claims, wherein S is greater than 0.

15. The CHP according to any one of the preceding claims, wherein the CHP is a first CHP, wherein the first CHP is bound to two other bioactive collagen hybridizing peptides to form a trimeric structure; and wherein the first CHP has a monomeric portion extending beyond the trimeric structure, the monomeric portion configured to bind to collagen alpha chains.

16. The CHP according to any one of claims 1-14, wherein the CHP is a first CHP, wherein the first CHP is bound to two other bioactive collagen hybridizing peptides to form a trimeric structure, and wherein a monomeric “Tail” CHP is conjugated to an N-terminal or a C-terminal of the first CHP of the trimeric structure using copper-free click chemistry or condensation reactions.

17. A composition comprising the CHP of any one of the preceding claims.

18. The composition according to claim 17, wherein each individual CHP does not form a triple helix with other CHPs.

19. The composition according to claim 17 or claim 18, further comprising a carrier.

20. The composition according to any one of claims 17-19, wherein the composition is a cosmetic composition, and the carrier comprises at least one selected from the group consisting of micelles, dendrites, lipids, microemulsions, nanoemulsions, solid lipid nanoparticles, polymers, gels, lens, surfactants, cyclodextrins, inserts, nanostructured lipid carriers, liposomes, transfersomes, ethosomes, niosomes, collagen matrix, extracellular matrix, and artificial extracellular matrix.

21. The composition according to any one of claims 17-20, wherein the composition is a topical cream, a saline solution, gel, polymer, or a solution for systemic injection within a subject.

22. The composition according to any one of claims 17-21, in which a CHP is bound directly to collagen matrix.

23. A method of reducing, preventing, or treating inflammation in a subject, comprising administering the CHP of any one of claims 1-16 or the composition of any one of claims 17-22.

24. The method according to claim 23, wherein the administering is performed by injection.

25. The method according to claim 23, wherein the administering is performed by micro-dermal injection.

26. The method according to claim 23, wherein the administering is performed by topical application.

27. A method of inhibiting or reducing an inflammatory response in a subject, comprising administering the CHP of any one of claims 1-16 or the composition of any one of claims 17-22.

28. The method according to claim 27, wherein the administering is performed by injection.

29. The method according to claim 27, wherein the administering is performed by micro-dermal injection.

30. The method according to claim 27, wherein the administering is performed by topical application.

31. A method of downregulating immune related cytokines including but not limited to; TLR ligands, interleukins, IFN-α, TLR-9, TNF-α, IL-1β, IL-6, CCL3, IL-16, IL-18, and/or TNF-α activity in a subject, comprising administering the CHP of any one of claims 1-16 or the composition of any one of claims 17-22.

32. A method of preventing the differentiation of immune cells comprising administering the CHP of any one of claims 1-16 or the composition of any one of claims 17-22.

33. A method of downregulating T-Cells, B-Cells, Natural Killer Cells, Macrophages, Monocytes, and Neutrophils comprising administering the CHP of any one of claims 1-16 or the composition of any one of claims 17-22.

34. A method of preventing the differentiation of T-Cells, B-Cells, Natural Killer Cells, Macrophages, Monocytes, and Neutrophils comprising administering the CHP of any one of claims 1-16 or the composition of any one of claims 17-22.

35. The method according to any one of claims 31-34, wherein the administering is performed by injection.

36. The method according to any one of claims 31-34, wherein the administering is performed by micro-dermal injection.

37. The method according to any one of claims 31-34, wherein the administering is performed by topical application.

38. A method of preventing or treating dry eye disease in a subject, comprising administering the CHP of any one of claims 1-16 or the composition of any one of claims 17-22.

39. The method according to claim 38, wherein the administering is performed by local injection, intravenous injection, topical application, or physical application.

40. The method according to claim 38, wherein the CHP or composition is administered as a putty, mesh, patch, adhesive, cream, sutures, gel or eyedrops.

41. A method of preventing or treating atopic dermatitis in a subject, comprising administering the CHP of any one of claims 1-16 or the composition of any one of claims 17-22.

42. The method according to claim 41, wherein the administering is performed by local injection, intravenous injection, topical application, or physical application.

43. The method according to claim 41, wherein the CHP or composition is administered as a putty, mesh, patch, adhesive, cream, sutures, or eyedrops.

44. A method of preventing or treating osteoarthritis in a subject, comprising administering the CHP of any one of claims 1-16 or the composition of any one of claims 17-22.

45. The method according to claim 44, wherein the administering is performed by local injection, intravenous injection, topical application, or physical application.

46. The method according to claim 44, wherein the CHP or composition is administered as a putty, mesh, patch, adhesive, cream, sutures, or eyedrops.

47. A method of preventing or treating rheumatoid arthritis in a subject, comprising administering the CHP of any one of claims 1-16 or the composition of any one of claims 17-22.

48. The method according to claim 47, wherein the administering is performed by local injection, intravenous injection, topical application, or physical application.

49. The method according to claim 47, wherein the CHP or composition is administered as a putty, mesh, patch, adhesive, cream, sutures, or eyedrops.

50. A method of preventing or treating diabetic lesions in a subject, comprising administering the CHP of any one of claims 1-16 or the composition of any one of claims 17-22.

51. The method according to claim 50, wherein the administering is performed by local injection, intravenous injection, topical application, or physical application.

52. The method according to claim 50, wherein the CHP or composition is administered as a putty, mesh, patch, adhesive, cream, sutures, or eyedrops.

53. A method of preventing or treating Psoriasis in a subject, comprising administering the CHP of any one of claims 1-16 or the composition of any one of claims 17-22.

54. The method according to claim 53, wherein the administering is performed by local injection, intravenous injection, topical application, or physical application.

55. The method according to claim 53, wherein the CHP or composition is administered as a putty, mesh, patch, adhesive, cream, sutures, or eyedrops.

56. A method of check point inhibition in a subject, comprising administering the CHP of any one of claims 1-16 or the composition of any one of claims 17-22.

57. The method according to claim 56, wherein the administering is performed by local injection, intravenous injection, topical application, or physical application.

58. The method according to claim 56, wherein the CHP or composition is administered as a putty, mesh, patch, adhesive, cream, sutures, or eyedrops.

59. A method of wound healing including diabetic lesions in a subject, comprising administering the CHP of any one of claims 1-16 or the composition of any one of claims 17-22.

60. The method according to claim 59, wherein the administering is performed by local injection, intravenous injection, topical application, or physical application.

61. The method according to claim 59, wherein the CHP or composition is administered as a putty, mesh, patch, adhesive, cream, sutures, or eyedrops.

62. A method of GI inflammatory conditions such as Crohn's and Ulcerative Colitis in a subject, comprising administering the CHP of any one of claims 1-16 or the composition of any one of claims 17-22.

63. The method according to claim 62, wherein the administering is performed by local injection, intravenous injection, topical application, or physical application.

64. The method according to claim 62, wherein the CHP or composition is administered as a putty, mesh, patch, adhesive, cream, sutures, or eyedrops.


SEQUENCE LISTING (106 sequences)

SEQ ID NO: 1 - protein, 16 aa, synthetic construct
  REPEAT 1..3: note = (GPP)x wherein x is any integer from 1-7
  REPEAT 14..16: note = (GPP)y wherein y is any integer from 1-7
  MOD_RES 4: note = X = hydroxyproline
  MOD_RES 1: note = An acetyl (capping) group at the N-terminus
GPPXGQDGLAGPKGPP

SEQ ID NO: 2 - protein, 15 aa, synthetic construct
  REPEAT 1..3: note = (GPX)x wherein x is any integer from 1-7
  REPEAT 13..15: note = (GPX)y wherein y is any integer from 1-7
  MOD_RES 13: note = X = hydroxyproline
  MOD_RES 3: note = X = hydroxyproline
  MOD_RES 1: note = An acetyl (capping) group at the N-terminus
GPXGQDGLAGPKGPX

SEQ ID NO: 3 - protein, 15 aa, synthetic construct
  REPEAT 1..3: note = (GXX)x wherein x is any integer from 1-7
  REPEAT 13..15: note = (GXX)y wherein y is any integer from 1-7
  MOD_RES 15: note = X = hydroxyproline
  MOD_RES 14: note = X = 2S,4S-4-fluoroproline
  MOD_RES 3: note = X = hydroxyproline
  MOD_RES 2: note = X = 2S,4S-4-fluoroproline
  MOD_RES 1: note = An acetyl (capping) group at the N-terminus
GXXGQDGLAGPKGXX

SEQ ID NO: 4 - protein, 16 aa, synthetic construct
  REPEAT 1..2: note = (GPP)x wherein x is any integer from 1-7
  REPEAT 14..16: note = (GPO)y wherein y is any integer from 1-7
  MOD_RES 16: note = X = hydroxyproline
  MOD_RES 4: note = X = hydroxyproline
  MOD_RES 1: note = An acetyl (capping) group at the N-terminus
GPPXGQDGLAGPKGPX

SEQ ID NO: 5 - protein, 15 aa, synthetic construct
  REPEAT 1..3: note = (GPX)x wherein x is any integer from 1-7
  REPEAT 13..15: note = (GPP)y wherein y is any integer from 1-7
  MOD_RES 3: note = X = hydroxyproline
  MOD_RES 1: note = An acetyl (capping) group at the N-terminus
GPXGQDGLAGPKGPP

SEQ ID NO: 6 - protein, 15 aa, synthetic construct
  REPEAT 1..3: note = (GPP)x wherein x is any integer from 1-7
  REPEAT 13..15: note = (GXX)y wherein y is any integer from 1-7
  MOD_RES 15: note = X = hydroxyproline
  MOD_RES 14: note = X = 2S,4S-4-fluoroproline
  MOD_RES 1: note = An acetyl (capping) group at the N-terminus
GPPGQDGLAGPKGXX

SEQ ID NO: 7 - protein, 16 aa, synthetic construct
  REPEAT 1..3: note = (GPX)x wherein x is any integer from 1-7
  REPEAT 14..16: note = (GXX)y wherein y is any integer from 1-7
  MOD_RES 16: note = X = hydroxyproline
  MOD_RES 15: note = X = 2S,4S-4-fluoroproline
  MOD_RES 3: note = X = hydroxyproline
  MOD_RES 1: note = An acetyl (capping) group at the N-terminus
GPXXGQDGLAGPKGXX

SEQ ID NO: 8 - protein, 15 aa, synthetic construct
  REPEAT 1..3: note = (GXX)x wherein x is any integer from 1-7
  REPEAT 13..15: note = (GPX)y wherein y is any integer from 1-7
  MOD_RES 15: note = X = hydroxyproline
  MOD_RES 3: note = X = hydroxyproline
  MOD_RES 2: note = X = 2S,4S-4-fluoroproline
  MOD_RES 1: note = An acetyl (capping) group at the N-terminus
GXXGQDGLAGPKGPX

SEQ ID NO: 9 - protein, 15 aa, synthetic construct
  REPEAT 1..3: note = (GXX)x wherein x is any integer from 1-7
  REPEAT 13..15: note = (GPP)y wherein y is any integer from 1-7
  MOD_RES 3: note = X = hydroxyproline
  MOD_RES 2: note = X = 2S,4S-4-fluoroproline
  MOD_RES 1: note = An acetyl (capping) group at the N-terminus
GXXGQDGLAGPKGPP

SEQ ID NO: 10 - protein, 12 aa, synthetic construct
  REPEAT 1..3: note = (GPP)x wherein x is any integer from 1-7
  REPEAT 10..12: note = (GPP)y wherein y is any integer from 1-7
  MOD_RES 1: note = An acetyl (capping) group at the N-terminus
GPPVTYAQLGPP

SEQ ID NO: 11 - protein, 12 aa, synthetic construct
  REPEAT 1..3: note = (GPP)x wherein x is any integer from 1-7
  REPEAT 10..12: note = (GPX)y wherein y is any integer from 1-7
  MOD_RES 12: note = X = hydroxyproline
  MOD_RES 1: note = An acetyl (capping) group at the N-terminus
GPPVTYAQLGPX

SEQ ID NO: 12 - protein, 12 aa, synthetic construct
  REPEAT 1..3: note = (GPP)x wherein x is any integer from 1-7
  REPEAT 10..12: note = (GXX)y wherein y is any integer from 1-7
  MOD_RES 12: note = X = hydroxyproline
  MOD_RES 11: note = X = 2S,4S-4-fluoroproline
  MOD_RES 1: note = An acetyl (capping) group at the N-terminus
GPPVTYAQLGXX

SEQ ID NO: 13 - protein, 12 aa, synthetic construct
  REPEAT 1..3: note = (GPX)x wherein x is any integer from 1-7
  REPEAT 10..12: note = (GPX)y wherein y is any integer from 1-7
  MOD_RES 12: note = X = hydroxyproline
  MOD_RES 3: note = X = hydroxyproline
  MOD_RES 1: note = An acetyl (capping) group at the N-terminus
GPXVTYAQLGPX

SEQ ID NO: 14 - protein, 12 aa, synthetic construct
  REPEAT 1..3: note = (GPX)x wherein x is any integer from 1-7
  REPEAT 10..12: note = (GPP)y wherein y is any integer from 1-7
  MOD_RES 3: note = X = hydroxyproline
  MOD_RES 1: note = An acetyl (capping) group at the N-terminus
GPOVTYAQLGPP

SEQ ID NO: 15 - protein, 12 aa, synthetic construct
  REPEAT 1..3: note = (GPX)x wherein x is any integer from 1-7
  REPEAT 10..12: note = (GXX)y wherein y is any integer from 1-7
  MOD_RES 12: note = X = hydroxyproline
  MOD_RES 11: note = X = 2S,4S-4-fluoroproline
  MOD_RES 3: note = X = hydroxyproline
  MOD_RES 1: note = X = hydroxyproline
GPXVTYAQLGXX

SEQ ID NO: 16 - protein, 12 aa, synthetic construct
  REPEAT 1..3: note = (GXX)x wherein x is any integer from 1-7
  MOD_RES 12: note = X = hydroxyproline
  MOD_RES 11: note = X = 2S,4S-4-fluoroproline
  MOD_RES 3: note = X = hydroxyproline
  MOD_RES 2: note = X = 2S,4S-4-fluoroproline
  MOD_RES 2: note = X = 2S,4S-4-fluoroproline
  MOD_RES 1: note = An acetyl (capping) group at the N-terminus
  REPEAT 10..12: note = (GXX)y wherein y is any integer from 1-7
GXXVTYAQLGXX

SEQ ID NO: 17 - protein, 12 aa, synthetic construct
  REPEAT 1..3: note = (GXX)x wherein x is any integer from 1-7
  REPEAT 10..12: note = (GPX)y wherein y is any integer from 1-7
  MOD_RES 12: note = X = hydroxyproline
  MOD_RES 3: note = X = hydroxyproline
  MOD_RES 2: note = X = 2S,4S-4-fluoroproline
  MOD_RES 1: note = An acetyl (capping) group at the N-terminus
GXXVTYAQLGPX

SEQ ID NO: 18 - protein, 12 aa, synthetic construct
  REPEAT 1..3: note = (GXX)x wherein x is any integer from 1-7
  REPEAT 10..12: note = (GPP)y wherein y is any integer from 1-7
  MOD_RES 3: note = X = hydroxyproline
  MOD_RES 2: note = X = 2S,4S-4-fluoroproline
  MOD_RES 1: note = An acetyl (capping) group at the N-terminus
GFXVTYAQLGPP

SEQ ID NO: 19 - protein, 12 aa, synthetic construct
  REPEAT 1..3: note = (GPP)x wherein x is any integer from 1-7
  MOD_RES 1: note = An acetyl (capping) group at the N-terminus
  REPEAT 10..12: note = (GPP)y wherein y is any integer from 1-7
GPPITYAAVGPP

SEQ ID NO: 20 - protein, 12 aa, synthetic construct
  REPEAT 1..3: note = (GPP)x wherein x is any integer from 1-7
  REPEAT 10..12: note = (GPX)y wherein y is any integer from 1-7
  MOD_RES 12: note = X = hydroxyproline
  MOD_RES 1: note = An acetyl (capping) group at the N-terminus
GPPITYAAVGPX

SEQ ID NO: 21 - protein, 12 aa, synthetic construct
  REPEAT 1..3: note = (GPP)x wherein x is any integer from 1-7
  REPEAT 10..12: note = (GXX)y wherein y is any integer from 1-7
  MOD_RES 12: note = X = hydroxyproline
  MOD_RES 11: note = X = 2S,4S-4-fluoroproline
  MOD_RES 1: note = An acetyl (capping) group at the N-terminus
GPPITYAAVGXX

SEQ ID NO: 22 - protein, 12 aa, synthetic construct
  REPEAT 1..3: note = (GPX)x wherein x is any integer from 1-7
  REPEAT 10..12: note = (GPX)y wherein y is any integer from 1-7
  MOD_RES 12: note = X = hydroxyproline
  MOD_RES 3: note = X = hydroxyproline
  MOD_RES 1: note = An acetyl (capping) group at the N-terminus
GPXITYAAVGPX

SEQ ID NO: 23 - protein, 12 aa, synthetic construct
  REPEAT 1..3: note = (GPX)x wherein x is any integer from 1-7
  REPEAT 10..12: note = (GPP)y wherein y is any integer from 1-7
  MOD_RES 3: note = X = hydroxyproline
  MOD_RES 1: note = An acetyl (capping) group at the N-terminus
GPXITYAAVGPP

SEQ ID NO: 24 - protein, 12 aa, synthetic construct
  REPEAT 1..3: note = (GPX)x wherein x is any integer from 1-7
  REPEAT 10..12: note = (GXX)y wherein y is any integer from 1-7
  MOD_RES 12: note = X = hydroxyproline
  MOD_RES 11: note = X = 2S,4S-4-fluoroproline
  MOD_RES 3: note = X = hydroxyproline
  MOD_RES 1: note = An acetyl (capping) group at the N-terminus
GPXITYAAVGXX

SEQ ID NO: 25 - protein, 12 aa, synthetic construct
  REPEAT 1..3: note = (GXX)x wherein x is any integer from 1-7
  REPEAT 10..12: note = (GXX)y wherein y is any integer from 1-7
  MOD_RES 12: note = X = hydroxyproline
  MOD_RES 11: note = X = 2S,4S-4-fluoroproline
  MOD_RES 3: note = X = hydroxyproline
  MOD_RES 2: note = X = 2S,4S-4-fluoroproline
  MOD_RES 1: note = An acetyl (capping) group at the N-terminus
GXXITYAAVGXX

SEQ ID NO: 26 - protein, 12 aa, synthetic construct
  REPEAT 1..3: note = (GXX)x wherein x is any integer from 1-7
  REPEAT 10..12: note = (GPP)y wherein y is any integer from 1-7
  MOD_RES 3: note = X = hydroxyproline
  MOD_RES 2: note = X = 2S,4S-4-fluoroproline
  MOD_RES 1: note = An acetyl (capping) group at the N-terminus
GXXITYAAVGPP

SEQ ID NO: 27 - protein, 12 aa, synthetic construct
  REPEAT 1..3: note = (GXX)x wherein x is any integer from 1-7
  REPEAT 10..12: note = (GPX)y wherein y is any integer from 1-7
  MOD_RES 12: note = X = hydroxyproline
  MOD_RES 3: note = X = hydroxyproline
  MOD_RES 2: note = X = 2S,4S-4-fluoroproline
  MOD_RES 1: note = An acetyl (capping) group at the N-terminus
GXXITYAAVGPX

SEQ ID NO: 28 - protein, 12 aa, synthetic construct
  REPEAT 1..3: note = (GPP)x wherein x is any integer from 1-7
  REPEAT 10..12: note = (GPP)y wherein y is any integer from 1-7
GPPLLYADLGPP

SEQ ID NO: 29 - protein, 12 aa, synthetic construct
  REPEAT 1..3: note = (GPP)x wherein x is any integer from 1-7
  REPEAT 10..12: note = (GPX)y wherein y is any integer from 1-7
  MOD_RES 12: note = X = hydroxyproline
  MOD_RES 1: note = An acetyl (capping) group at the N-terminus
GPPLLYADLGPX

SEQ ID NO: 30 - protein, 12 aa, synthetic construct
  REPEAT 1..3: note = (GPP)x wherein x is any integer from 1-7
  REPEAT 10..12: note = (GXX)y wherein y is any integer from 1-7
  MOD_RES 12: note = X = hydroxyproline
  MOD_RES 11: note = X = 2S,4S-4-fluoroproline
  MOD_RES 1: note = An acetyl (capping) group at the N-terminus
GPPLLYADLGXX

SEQ ID NO: 31 - protein, 12 aa, synthetic construct
  REPEAT 1..3: note = (GPX)x wherein x is any integer from 1-7
  REPEAT 10..12: note = (GPX)y wherein y is any integer from 1-7
  MOD_RES 12: note = X = hydroxyproline
  MOD_RES 3: note = X = hydroxyproline
  MOD_RES 1: note = An acetyl (capping) group at the N-terminus
GPXLLYADLGPX

SEQ ID NO: 32 - protein, 12 aa, synthetic construct
  REPEAT 1..3: note = (GPX)x wherein x is any integer from 1-7
  REPEAT 10..12: note = (GPP)y wherein y is any integer from 1-7
  MOD_RES 3: note = X = hydroxyproline
  MOD_RES 1: note = An acetyl (capping) group at the N-terminus
GPXLLYADLGPP

SEQ ID NO: 33 - protein, 12 aa, synthetic construct
  REPEAT 1..3: note = (GPX)x wherein x is any integer from 1-7
  REPEAT 10..12: note = (GXX)y wherein y is any integer from 1-7
  MOD_RES 12: note = X = hydroxyproline
  MOD_RES 11: note = X = 2S,4S-4-fluoroproline
  MOD_RES 3: note = X = hydroxyproline
  MOD_RES 1: note = An acetyl (capping) group at the N-terminus
GPXLLYADLGXX

SEQ ID NO: 34 - protein, 12 aa, synthetic construct
  REPEAT 1..3: note = (GXX)x wherein x is any integer from 1-7
  REPEAT 10..12: note = (GXX)y wherein y is any integer from 1-7
  MOD_RES 12: note = X = hydroxyproline
  MOD_RES 11: note = X = 2S,4S-4-fluoroproline
  MOD_RES 3: note = X = hydroxyproline
  MOD_RES 2: note = X = 2S,4S-4-fluoroproline
  MOD_RES 1: note = An acetyl (capping) group at the N-terminus
GXXLLYADLGXX

SEQ ID NO: 35 - protein, 12 aa, synthetic construct
  REPEAT 1..3: note = (GXX)x wherein x is any integer from 1-7
  REPEAT 10..12: note = (GPP)y wherein y is any integer from 1-7
  MOD_RES 3: note = X = hydroxyproline
  MOD_RES 2: note = X = 2S,4S-4-fluoroproline
  MOD_RES 1: note = An acetyl (capping) group at the N-terminus
GXXLLYADLGPP

SEQ ID NO: 36 - protein, 12 aa, synthetic construct
  REPEAT 1..3: note = (GXX)x wherein x is any integer from 1-7
  REPEAT 10..12: note = (GPX)y wherein y is any integer from 1-7
  MOD_RES 12: note = X = hydroxyproline
  MOD_RES 3: note = X = hydroxyproline
  MOD_RES 2: note = X = 2S,4S-4-fluoroproline
  MOD_RES 1: note = An acetyl (capping) group at the N-terminus
GXXLLYADLGPX

SEQ ID NO: 37 - protein, 12 aa, synthetic construct
  REPEAT 1..2: note = (GXX)x wherein x is any integer from 1-7
  REPEAT 10..12: note = (GXX)y wherein y is any integer from 1-7
  MOD_RES 12: note = X = hydroxyproline
  MOD_RES 11: note = X = 2S,4S-4-fluoroproline
  MOD_RES 3: note = X = hydroxyproline
  MOD_RES 2: note = X = 2S,4S-4-fluoroproline
  MOD_RES 1: note = An acetyl (capping) group at the N-terminus
GXXLLYADLGXX

SEQ ID NO: 38 - protein, 12 aa, synthetic construct
  REPEAT 1..3: note = (GPP)x wherein x is any integer from 1-7
  REPEAT 10..12: note = (GPP)y wherein y is any integer from 1-7
  MOD_RES 1: note = An acetyl (capping) group at the N-terminus
GPPTIYAVVGPP

SEQ ID NO: 39 - protein, 12 aa, synthetic construct
  REPEAT 1..3: note = (GPP)x wherein x is any integer from 1-7
  REPEAT 10..12: note = (GPX)y wherein y is any integer from 1-7
  MOD_RES 12: note = X = hydroxyproline
  MOD_RES 1: note = An acetyl (capping) group at the N-terminus
GPPTIYAVVGPX

SEQ ID NO: 40 - protein, 12 aa, synthetic construct
  REPEAT 1..3: note = (GPP)x wherein x is any integer from 1-7
  REPEAT 10..12: note = (GXX)y wherein y is any integer from 1-7
  MOD_RES 12: note = X = hydroxyproline
  MOD_RES 11: note = X = 2S,4S-4-fluoroproline
  MOD_RES 1: note = An acetyl (capping) group at the N-terminus
GPPTIYAVVGXX

SEQ ID NO: 41 - protein, 12 aa, synthetic construct
  REPEAT 1..3: note = (GPX)x wherein x is any integer from 1-7
  REPEAT 10..12: note = (GPX)y wherein y is any integer from 1-7
  MOD_RES 12: note = X = hydroxyproline
  MOD_RES 3: note = X = hydroxyproline
  MOD_RES 1: note = An acetyl (capping) group at the N-terminus
GPXTIYAVVGPX

SEQ ID NO: 42 - protein, 12 aa, synthetic construct
  REPEAT 1..3: note = (GPX)x wherein x is any integer from 1-7
  REPEAT 10..12: note = (GPP)y wherein y is any integer from 1-7
  MOD_RES 3: note = X = hydroxyproline
  MOD_RES 1: note = An acetyl (capping) group at the N-terminus
GPXTIYAVVGPP

SEQ ID NO: 43 - protein, 12 aa, synthetic construct
  REPEAT 1..3: note = (GPX)x wherein x is any integer from 1-7
  REPEAT 10..12: note = (GXX)y wherein y is any integer from 1-7
  MOD_RES 12: note = X = hydroxyproline
  MOD_RES 11: note = X = 2S,4S-4-fluoroproline
  MOD_RES 3: note = X = hydroxyproline
  MOD_RES 1: note = An acetyl (capping) group at the N-terminus
GPXTIYAVVGXX

SEQ ID NO: 44 - protein, 12 aa, synthetic construct
  REPEAT 1..3: note = (GXX)x wherein x is any integer from 1-7
  REPEAT 10..12: note = (GXX)y wherein y is y is any integer from 1-7
  MOD_RES 12: note = X = hydroxyproline
  MOD_RES 11: note = X = 2S,4S-4-fluoroproline
  MOD_RES 3: note = X = hydroxyproline
  MOD_RES 2: note = X = 2S,4S-4-fluoroproline
  MOD_RES 1: note = An acetyl (capping) group at the N-terminus
GXXTIYAVVGXX

SEQ ID NO: 45 - protein, 12 aa, synthetic construct
  REPEAT 1..3: note = (GXX)x wherein x is any integer from 1-7
  REPEAT 10..12: note = (GPP)y wherein y is any integer from 1-7
  MOD_RES 3: note = X = hydroxyproline
  MOD_RES 2: note = X = 2S,4S-4-fluoroproline
  MOD_RES 1: note = An acetyl (capping) group at the N-terminus
GXXTIYAVVGPP

SEQ ID NO: 46 - protein, 12 aa, synthetic construct
  REPEAT 1..3: note = (GXX)x wherein x is any integer from 1-7
  REPEAT 1..12: note = (GPX)y wherein y is any integer from 1-7
  MOD_RES 12: note = X = hydroxyproline
  MOD_RES 3: note = X = hydroxyproline
  MOD_RES 2: note = X = 2S,4S-4-fluoroproline
  MOD_RES 1: note = An acetyl (capping) group at the N-terminus
GXXTIYAVVGPX

SEQ ID NO: 47 - protein, 12 aa, synthetic construct
  REPEAT 1..3: note = (GPP)x wherein x is any integer from 1-7
  REPEAT 10..12: note = (GPP)y wherein y is any integer from 1-7
  MOD_RES 1: note = An acetyl (capping) group at the N-terminus
GPPITYSLLGPP

SEQ ID NO: 48 - protein, 12 aa, synthetic construct
  REPEAT 1..3: note = (GPP)x wherein x is any integer from 1-7
  REPEAT 10..12: note = (GPX)y wherein y is any integer from 1-7
  MOD_RES 12: note = X = hydroxyproline
  MOD_RES 1: note = An acetyl (capping) group at the N-terminus
GPPITYSLLGPX

SEQ ID NO: 49 - protein, 12 aa, synthetic construct
  REPEAT 1..3: note = (GPP)x wherein x is any integer from 1-7
  REPEAT 10..12: note = (GXX)y wherein y is any integer from 1-7
  MOD_RES 12: note = X = hydroxyproline
  MOD_RES 11: note = X = 2S,4S-4-fluoroproline
  MOD_RES 1: note = An acetyl (capping) group at the N-terminus
GPPITYSLLGXX

SEQ ID NO: 50 - protein, 12 aa, synthetic construct
  REPEAT 1..3: note = (GPX)x wherein x is any integer from 1-7
  REPEAT 10..12: note = (GPX)y wherein y is any integer from 1-7
  MOD_RES 12: note = X = hydroxyproline
  MOD_RES 3: note = X = hydroxyproline
  MOD_RES 1: note = An acetyl (capping) group at the N-terminus
GPXITYSLLGPX

SEQ ID NO: 51 - protein, 12 aa, synthetic construct
  REPEAT 1..3: note = (GPX)x wherein x is any integer from 1-7
  REPEAT 10..12: note = (GPP)y wherein y is any integer from 1-7
  MOD_RES 3: note = X = hydroxyproline
  MOD_RES 1: note = An acetyl (capping) group at the N-terminus
GPXITYSLLGPP

SEQ ID NO: 52 - protein, 12 aa, synthetic construct
  REPEAT 1..3: note = (GPX)x wherein x is any integer from 1-7
  REPEAT 10..12: note = (GXX)y wherein y is any integer from 1-7
  MOD_RES 12: note = X = hydroxyproline
  MOD_RES 11: note = X = 2S,4S-4-fluoroproline
  MOD_RES 3: note = X = hydroxyproline
  MOD_RES 1: note = An acetyl (capping) group at the N-terminus
GPXITYSLLGXX

SEQ ID NO: 53 - protein, 12 aa, synthetic construct
  REPEAT 1..3: note = (GXX)x wherein x is any integer from 1-7
  REPEAT 10..12: note = (GXX)y wherein y is any integer from 1-7
  MOD_RES 12: note = X = hydroxyproline
  MOD_RES 11: note = X = 2S,4S-4-fluoroproline
  MOD_RES 3: note = X = hydroxyproline
  MOD_RES 2: note = X = 2S,4S-4-fluoroproline
  MOD_RES 1: note = An acetyl (capping) group at the N-terminus
GXXITYSLLGXX

SEQ ID NO: 54 - protein, 12 aa, synthetic construct
  REPEAT 1..3: note = (GXX)x wherein x is any integer from 1-7
  REPEAT 10..12: note = (GPP)y wherein y is any integer from 1-7
  MOD_RES 3: note = X = hydroxyproline
  MOD_RES 2: note = X = 2S,4S-4-fluoroproline
  MOD_RES 1: note = An acetyl (capping) group at the N-terminus
GXXITYSLLGPP

SEQ ID NO: 55 - protein, 12 aa, synthetic construct
  REPEAT 1..3: note = GfO)x- ITYSLL-(GPO
  REPEAT 10..12: note = (GPX)y wherein y is any integer from 1-7
  MOD_RES 12: note = X = hydroxyproline
  MOD_RES 3: note = X = hydroxyproline
  MOD_RES 2: note = X = 2S,4S-4-fluoroproline
  MOD_RES 1: note = An acetyl (capping) group at the N-terminus
GXXITYSLLGPX

SEQ ID NO: 56 - protein, 33 aa, synthetic construct
  REPEAT 1..3: note = (GPP)x wherein x is any integer from 1-7
  REPEAT 31..33: note = (GPP)y wherein y is any integer from 1-7
  MOD_RES 6: note = X = hydroxyproline
  MOD_RES 15: note = X = hydroxyproline
  MOD_RES 27: note = X = hydroxyproline
  MOD_RES 30: note = X = hydroxyproline
  MOD_RES 1: note = An acetyl (capping) group at the N-terminus
GPPGAXGLRGGAGPXGPEGGKGAAGPXGPXGPP

SEQ ID NO: 57 - protein, 33 aa, synthetic construct
  REPEAT 1..3: note = (GPP)x wherein x is any integer from 1-7
  REPEAT 31..33: note = (GPX)y wherein y is any integer from 1-7
  MOD_RES 6: note = X = hydroxyproline
  MOD_RES 15: note = X = hydroxyproline
  MOD_RES 27: note = X = hydroxyproline
  MOD_RES 30: note = X = hydroxyproline
  MOD_RES 33: note = X = hydroxyproline
  MOD_RES 1: note = An acetyl (capping) group at the N-terminus
GPPGAXGLRGGAGPXGPEGGKGAAGPXGPXGPX

SEQ ID NO: 58 - protein, 33 aa, synthetic construct
  REPEAT 1..3: note = (GPP)x wherein x is any integer from 1-7
  REPEAT 31..33: note = (GXX)y wherein y is any integer from 1-7
  MOD_RES 6: note = X = hydroxyproline
  MOD_RES 15: note = X = hydroxyproline
  MOD_RES 27: note = X = hydroxyproline
  MOD_RES 30: note = X = hydroxyproline
  MOD_RES 33: note = X = hydroxyproline
  MOD_RES 32: note = X = 2S,4S-4-fluoroproline
  MOD_RES 1: note = An acetyl (capping) group at the N-terminus
GPPGAXGLRGGAGPXGPEGGKGAAGPXGPXGXX

SEQ ID NO: 59 - protein, 33 aa, synthetic construct
  REPEAT 1..3: note = (GPX)x wherein x is any integer from 1-7
  REPEAT 31..33: note = (GPX)y wherein y is any integer from 1-7
  MOD_RES 3: note = X = hydroxyproline
  MOD_RES 6: note = X = hydroxyproline
  MOD_RES 15: note = X = hydroxyproline
  MOD_RES 27: note = X = hydroxyproline
  MOD_RES 30: note = X = hydroxyproline
  MOD_RES 33: note = X = hydroxyproline
GPXGAXGLRGGAGPXGPEGGKGAAGPXGPXGPX

SEQ ID NO: 60 - protein, 33 aa, synthetic construct
  REPEAT 1..3: note = (GPX)x wherein x is any integer from 1-7
  REPEAT 31..33: note = (GPP)y wherein y is any integer from 1-7
  MOD_RES 3: note = X = hydroxyproline
  MOD_RES 6: note = X = hydroxyproline
  MOD_RES 15: note = X = hydroxyproline
  MOD_RES 27: note = X = hydroxyproline
  MOD_RES 30: note = X = hydroxyproline
  MOD_RES 1: note = An acetyl (capping) group at the N-terminus
GPXGAXGLRGGAGPXGPEGGKGAAGPXGPXGPP

SEQ ID NO: 61 - protein, 33 aa, synthetic construct
  REPEAT 1..3: note = (GPX)x wherein x is any integer from 1-7
  REPEAT 31..33: note = (GXX)y wherein y is any integer from 1-7
  MOD_RES 3: note = X = hydroxyproline
  MOD_RES 6: note = X = hydroxyproline
  MOD_RES 15: note = X = hydroxyproline
  MOD_RES 27: note = X = hydroxyproline
  MOD_RES 30: note = X = hydroxyproline
  MOD_RES 32: note = X = 2S,4S-4-fluoroproline
  MOD_RES 33: note = X = hydroxyproline
  MOD_RES 1: note = An acetyl (capping) group at the N-terminus
GPXGAXGLRGGAGPXGPEGGKGAAGPXGPXGXX

SEQ ID NO: 62 - protein, 33 aa, synthetic construct
  REPEAT 1..3: note = (GXX)x wherein x is any integer from 1-7
  REPEAT 31..33: note = (GXX)y wherein y is any integer from 1-7
  MOD_RES 2: note = 2S,4S-4-fluoroproline
  MOD_RES 3: note = X = hydroxyproline
  MOD_RES 6: note = X = hydroxyproline
  MOD_RES 15: note = X = hydroxyproline
  MOD_RES 27: note = X = hydroxyproline
  MOD_RES 30: note = X = hydroxyproline
  MOD_RES 32: note = X = 2S,4S-4-fluoroproline
  MOD_RES 33: note = X = hydroxyproline
  MOD_RES 1: note = An acetyl (capping) group at the N-terminus
GXXGAXGLRGGAGPXGPEGGKGAAGPXGPXGPX

SEQ ID NO: 63 - protein, 33 aa, synthetic construct; identical to SEQ ID NO: 62
  REPEAT 1..3: note = (GXX)x wherein x is any integer from 1-7
  REPEAT 31..33: note = (GPX)y wherein y is any integer from 1-7
  MOD_RES 2: note = X = 2S,4S-4-fluoroproline
  MOD_RES 3: note = X = hydroxyproline
  MOD_RES 3: note = X = hydroxyproline
  MOD_RES 6: note = X = hydroxyproline
  MOD_RES 15: note = X = hydroxyproline
  MOD_RES 27: note = X = hydroxyproline
  MOD_RES 30: note = X = hydroxyproline
  MOD_RES 33: note = X = hydroxyproline

SEQ ID NO: 64 - protein, 33 aa, synthetic construct
  REPEAT 1..3: note = (GXX)x wherein x is any integer from 1-7
  REPEAT 31..33: note = (GPP)y wherein y is any integer from 1-7
  MOD_RES 2: note = X = 2S,4S-4-fluoroproline
  MOD_RES 3: note = X = hydroxyproline
  MOD_RES 6: note = X = hydroxyproline
  MOD_RES 15: note = X = hydroxyproline
  MOD_RES 27: note = X = hydroxyproline
  MOD_RES 30: note = X = hydroxyproline
  MOD_RES 1: note = An acetyl (capping) group at the N-terminus
GXXGAXGLRGGAGPXGPEGGKGAAGPXGPXGPP

SEQ ID NO: 65 - protein, 12 aa, synthetic construct
  REPEAT 1..3: note = (GPP)x wherein x is any integer from 1-7
  REPEAT 10..12: note = (GPP)y wherein y is any integer from 1-7
  MOD_RES 4: note = X = hydroxyproline
  MOD_RES 7: note = X = hydroxyproline
  MOD_RES 1: note = An acetyl (capping) group at the N-terminus
GPPXGPXGPGPP

SEQ ID NO: 66 - protein, 12 aa, synthetic construct
  REPEAT 1..3: note = (GPP)x wherein x is any integer from 1-7
  REPEAT 10..12: note = (GPX)y wherein y is any integer from 1-7
  MOD_RES 4: note = X = hydroxyproline
  MOD_RES 7: note = X = hydroxyproline
  MOD_RES 1: note = An acetyl (capping) group at the N-terminus
GPPXGPXGPGPX

SEQ ID NO: 67 - protein, 12 aa, synthetic construct
  REPEAT 1..3: note = (GPP)x wherein x is any integer from 1-7
  REPEAT 10..12: note = (GXX)y wherein y is any integer from 1-7
  MOD_RES 4: note = X = hydroxyproline
  MOD_RES 7: note = X = hydroxyproline
  MOD_RES 11: note = X = 2S,4S-4-fluoroproline
  MOD_RES 12: note = X = hydroxyproline
  MOD_RES 1: note = An acetyl (capping) group at the N-terminus
GPPXGPXGPGXX

SEQ ID NO: 68 - protein, 12 aa, synthetic construct
  REPEAT 1..3: note = (GPX)x wherein x is any integer from 1-7
  REPEAT 10..12: note = (GPX)y wherein y is any integer from 1-7
  MOD_RES 3: note = X = hydroxyproline
  MOD_RES 4: note = X = hydroxyproline
  MOD_RES 7: note = X = hydroxyproline
  MOD_RES 12: note = X = hydroxyproline
  MOD_RES 1: note = An acetyl (capping) group at the N-terminus
GPXXGPXGPGPX

SEQ ID NO: 69 - protein, 12 aa, synthetic construct
  REPEAT 1..3: note = (GPX)x wherein x is any integer from 1-7
  REPEAT 10..12: note = (GPP)y wherein y is any integer from 1-7
  MOD_RES 3: note = X = hydroxyproline
  MOD_RES 4: note = X = hydroxyproline
  MOD_RES 7: note = X = hydroxyproline
  MOD_RES 1: note = An acetyl (capping) group at the N-terminus
GPXXGPXGPGPP

SEQ ID NO: 70 - protein, 12 aa, synthetic construct
  REPEAT 1..3: note = (GPX)x wherein x is any integer from 1-7
  REPEAT 10..12: note = (GPX)x wherein x is any integer from 1-7
  MOD_RES 3: note = X = hydroxyproline
  MOD_RES 4: note = X = hydroxyproline
  MOD_RES 7: note = X = hydroxyproline
  MOD_RES 11: note = X = 2S,4S-4-fluoroproline
  MOD_RES 12: note = X = hydroxyproline
  MOD_RES 1: note = An acetyl (capping) group at the N-terminus
GPXXGPXGPGXX

SEQ ID NO: 71 - protein, 12 aa, synthetic construct
  REPEAT 1..3: note = (GXX)x wherein x is any integer from 1-7
  REPEAT 10..12: note = (GXX)y wherein y is any integer from 1-7
  MOD_RES 2: note = X = 2S,4S-4-fluoroproline
  MOD_RES 3: note = X = hydroxyproline
  MOD_RES 4: note = X = hydroxyproline
  MOD_RES 7: note = X = hydroxyproline
  MOD_RES 11: note = X = 2S,4S-4-fluoroproline
  MOD_RES 12: note = X = hydroxyproline
  MOD_RES 1: note = An acetyl (capping) group at the N-terminus
GXXXGPXGPGXX

SEQ ID NO: 72 - protein, 12 aa, synthetic construct
  REPEAT 1..3: note = (GXX)x wherein x is any integer from 1-7
  REPEAT 10..12: note = (GPX)y wherein y is any integer from 1-7
  MOD_RES 2: note = X = 2S,4S-4-fluoroproline
  MOD_RES 3: note = X = hydroxyproline
  MOD_RES 4: note = X = hydroxyproline
  MOD_RES 7: note = X = hydroxyproline
  MOD_RES 12: note = X = hydroxyproline
  MOD_RES 1: note = An acetyl (capping) group at the N-terminus
GXXXGPXGPGPX

SEQ ID NO: 73 - protein, 12 aa, synthetic construct
  REPEAT 1..3: note = (GXX)x wherein x is any integer from 1-7
  REPEAT 10..12: note = (GPP)y wherein y is any integer from 1-7
  MOD_RES 2: note = X = 2S,4S-4-fluoroproline
  MOD_RES 3: note = X = hydroxyproline
  MOD_RES 4: note = X = hydroxyproline
  MOD_RES 7: note = X = hydroxyproline
  MOD_RES 1: note = An acetyl (capping) group at the N-terminus
GXXXGPXGPGPP

SEQ ID NO: 74 - protein, 12 aa, synthetic construct
  REPEAT 1..3: note = (GPX)x wherein x is any integer from 1-7
  REPEAT 10..12: note = (GPX)y wherein y is any integer from 1-7
  MOD_RES 7: note = X = hydroxyproline
  MOTIF 1: note = An acetyl (capping) group at the N-terminus
GPPAGPXGPGPP

SEQ ID NO: 75 - protein, 12 aa, synthetic construct
  REPEAT 1..3: note = (GPP)x wherein x is any integer from 1-7
  REPEAT 10..12: note = (GPX)y wherein y is any integer from 1-7
  MOD_RES 7: note = X = hydroxyproline
  MOD_RES 12: note = X = hydroxyproline
  MOD_RES 1: note = An acetyl (capping) group at the N-terminus
GPPAGPXGPGPX

SEQ ID NO: 76 - protein, 12 aa, synthetic construct
  REPEAT 1..3: note = (GPP)x wherein x is any integer from 1-7
  REPEAT 10..12: note = (GXX)y wherein y is any integer from 1-7
  MOD_RES 7: note = X = hydroxyproline
  MOD_RES 11: note = X = 2S,4S-4-fluoroproline
  MOD_RES 12: note = X = hydroxyproline
  MOD_RES 1: note = An acetyl (capping) group at the N-terminus
GPPAGPXGPGXX

SEQ ID NO: 77 - protein, 12 aa, synthetic construct
  REPEAT 1..3: note = (GPX)x wherein x is any integer from 1-7
  REPEAT 10..12: note = (GPX)y wherein y is any integer from 1-7
  MOD_RES 3: note = X = hydroxyproline
  MOD_RES 7: note = X = hydroxyproline
  MOD_RES 12: note = X = hydroxyproline
  MOD_RES 1: note = An acetyl (capping) group at the N-terminus
GPXAGPXGPGPX

SEQ ID NO: 78 - protein, 12 aa, synthetic construct
  REPEAT 1..3: note = (GPX)x wherein x is any integer from 1-7
  REPEAT 10..12: note = (GPP)y wherein y is any integer from 1-7
  MOD_RES 3: note = X = hydroxyproline
  MOD_RES 7: note = X = hydroxyproline
  MOD_RES 1: note = An acetyl (capping) group at the N-terminus
GPXAGPXGPGPP

SEQ ID NO: 79 - protein, 12 aa, synthetic construct
  REPEAT 1..3: note = (GPX)x wherein x is any integer from 1-7
  REPEAT 10..12: note = (GXX)y wherein y is any integer from 1-7
  MOD_RES 3: note = X = hydroxyproline
  MOD_RES 7: note = X = hydroxyproline
  MOD_RES 11: note = X = hydroxyproline
  MOD_RES 12: note = X = hydroxyproline
  MOD_RES 1: note = An acetyl (capping) group at the N-terminus
GPXAGPXGPGXX

SEQ ID NO: 80 - protein, 12 aa, synthetic construct
  REPEAT 1..3: note = (GXX)x wherein x is any integer from 1-7
  REPEAT 10..12: note = (GXX)y wherein y is any integer from 1-7
  MOD_RES 3: note = X = 2S,4S-4-fluoroproline
  MOD_RES 2: note = X = hydroxyproline
  MOD_RES 7: note = X = hydroxyproline
  MOD_RES 11: note = X = 2S,4S-4-fluoroproline
  MOD_RES 12: note = X = hydroxyproline
  MOD_RES 1: note = An acetyl (capping) group at the N-terminus
GXXAGPXGPGXX

SEQ ID NO: 81 - protein, 12 aa, synthetic construct
  REGION 1..3: note = (GXX)x wherein x is any integer from 1-7
  REPEAT 10..12: note = (GPX)y wherein y is any integer from 1-7
  MOD_RES 2: note = X = 2S,4S-4-fluoroproline
  MOD_RES 3: note = X = hydroxyproline
  MOD_RES 7: note = X = hydroxyproline
  MOD_RES 12: note = X = hydroxyproline
  MOD_RES 1: note = An acetyl (capping) group at the N-terminus
GXXAGPXGPGPX

SEQ ID NO: 82 - protein, 12 aa, synthetic construct
  REPEAT 1..3: note = (GXX)x wherein x is any integer from 1-7
  REPEAT 10..12: note = (GPP)y wherein y is any integer from 1-7
  MOD_RES 2: note = X = 2S,4S-4-fluoroproline
  MOD_RES 3: note = X = hydroxyproline
  MOD_RES 7: note = X = hydroxyproline
  MOD_RES 1: note = An acetyl (capping) group at the N-terminus
GXXAGPXGPGPP

SEQ ID NO: 83 - protein, 15 aa, synthetic construct
  MOD_RES 1: note = An acetyl (capping) group at the N-terminus
GPOGPOGPOGPOGPO

SEQ ID NO: 84 - protein, 15 aa, synthetic construct
  REPEAT 1..3: note = (GPP)x wherein x is any integer from 1-7
  REPEAT 13..15: note = (GPP)y wherein y is any integer from 1-7
  MOD_RES 6: note = X = hydroxyproline
  MOD_RES 12: note = X = hydroxyproline
  MOD_RES 1: note = An acetyl (capping) group at the N-terminus
GPPGPXGPAGFXGPP

SEQ ID NO: 85 - protein, 15 aa, synthetic construct
  REPEAT 1..3: note = (GPP)x wherein x is any integer from 1-7
  REPEAT 13..15: note = (GPX)y wherein y is any integer from 1-7
  MOD_RES 6: note = X = hydroxyproline
  MOD_RES 12: note = X = hydroxyproline
  MOD_RES 15: note = X = hydroxyproline
  MOD_RES 1: note = An acetyl (capping) group at the N-terminus
GPPGPXGPAGFXGPX

SEQ ID NO: 86 - protein, 15 aa, synthetic construct
  REPEAT 1..3: note = (GPP)x wherein x is any integer from 1-7
  REPEAT 13..15: note = (GXX)y wherein y is any integer from 1-7
  MOD_RES 6: note = X = hydroxyproline
  MOD_RES 12: note = X = hydroxyproline
  MOD_RES 14: note = X = 2S,4S-4-fluoroproline
  MOD_RES 15: note = X = hydroxyproline
  MOD_RES 1: note = An acetyl (capping) group at the N-terminus
GPPGPXGPAGFXGXX

SEQ ID NO: 87 - protein, 15 aa, synthetic construct
  REPEAT 1..3: note = (GPX)x wherein x is any integer from 1-7
  REPEAT 13..15: note = (GPX)y wherein y is any integer from 1-7
  MOD_RES 3: note = X = hydroxyproline
  MOD_RES 6: note = X = hydroxyproline
  MOD_RES 12: note = X = hydroxyproline
  MOD_RES 15: note = X = hydroxyproline
  MOD_RES 1: note = An acetyl (capping) group at the N-terminus
GPXGPXGPAGFXGPX

SEQ ID NO: 88 - protein, 15 aa, synthetic construct
  REPEAT 1..3: note = (GPX)x wherein x is any integer from 1-7
  REPEAT 10..13: note = (GPP)y wherein y is any integer from 1-7
  MOD_RES 6: note = X = hydroxyproline
  MOD_RES 3: note = X = hydroxyproline
  MOD_RES 12: note = X = hydroxyproline
  MOD_RES 1: note = An acetyl (capping) group at the N-terminus
GPXGPXGPAGFXGPP

SEQ ID NO: 89 - protein, 15 aa, synthetic construct
  REPEAT 1..3: note = (GPX)x wherein x is any integer from 1-7
  REPEAT 13..15: note = (GXX)y wherein y is any integer from 1-7
  MOD_RES 3: note = X = hydroxyproline
  MOD_RES 6: note = X = hydroxyproline
  MOD_RES 12: note = X = hydroxyproline
  MOD_RES 14: note = X = 2S,4S-4-fluoroproline
  MOD_RES 15: note = X = hydroxyproline
  MOD_RES 1: note = An acetyl (capping) group at the N-terminus
GPXGPXGPAGFXGXX

SEQ ID NO: 90 - protein, 15 aa, synthetic construct
  REPEAT 1..3: note = (GXX)x wherein x is any integer from 1-7
  REPEAT 13..15: note = (GXX)y wherein y is any integer from 1-7
  MOD_RES 2: note = X = 2S,4S-4-fluoroproline
  MOD_RES 3: note = X = hydroxyproline
  MOD_RES 6: note = X = hydroxyproline
  MOD_RES 12: note = X = hydroxyproline
  MOD_RES 14: note = X = 2S,4S-4-fluoroproline
  MOD_RES 15: note = X = hydroxyproline
  MOD_RES 1: note = An acetyl (capping) group at the N-terminus
GXXGPXGPAGFXGXX

SEQ ID NO: 91 - protein, 15 aa, synthetic construct
  REPEAT 1..3: note = (GXX)x wherein x is any integer from 1-7
  REPEAT 13..15: note = (GPX)y wherein y is any integer from 1-7
  MOD_RES 2: note = X = 2S,4S-4-fluoroproline
  MOD_RES 3: note = X = hydroxyproline
  MOD_RES 6: note = X = hydroxyproline
  MOD_RES 12: note = X = hydroxyproline
  MOD_RES 15: note = X = hydroxyproline
  MOD_RES 1: note = An acetyl (capping) group at the N-terminus
GXXGPXGPAGFXGPX

SEQ ID NO: 92 - protein, 15 aa, synthetic construct
  REPEAT 1..3: note = (GXX)x wherein x is any integer from 1-7
  REPEAT 13..15: note = (GPP)y wherein y is any integer from 1-7
  MOD_RES 2: note = X = 2S,4S-4-fluoroproline
  MOD_RES 3: note = X = hydroxyproline
  MOD_RES 6: note = X = hydroxyproline
  MOD_RES 1: note = An acetyl (capping) group at the N-terminus
GXXGPXGPAGFXGPP

SEQ ID NO: 93 - protein, 6 aa, synthetic construct
VTYAQL

SEQ ID NO: 94 - protein, 6 aa, synthetic construct
ITYAAV

SEQ ID NO: 95 - protein, 6 aa, synthetic construct
LLYADL

SEQ ID NO: 96 - protein, 6 aa, synthetic construct
TIYAVV

SEQ ID NO: 97 - protein, 6 aa, synthetic construct
ITYSLL

SEQ ID NO: 98 - protein, 27 aa, synthetic construct
GAOGLRGGAGPOGPEGGKGAAGPOGPO

SEQ ID NO: 99 - protein, 9 aa, synthetic construct
GQDGLAGPK

SEQ ID NO: 100 - protein, 10 aa, synthetic construct
OGQDGLAGPK

SEQ ID NO: 101 - protein, 6 aa, synthetic construct
OGPOGP

SEQ ID NO: 102 - protein, 6 aa, synthetic construct
AGPOGP

SEQ ID NO: 103 - protein, 15 aa, synthetic construct
GPOGPOGPOGPOGPO

SEQ ID NO: 104 - protein, 9 aa, synthetic construct
GPOGPAGFO

SEQ ID NO: 105 - protein, 6 aa, synthetic construct
  REPEAT 1..3: note = (GGG)z wherein z is from 1-3
GGGGGG

SEQ ID NO: 106 - protein, 30 aa, synthetic construct
  MOD_RES 1: note = Biotin is attached
GGGGPOGPOGPOGPOGPOGPOGPOGPOGPO